(19) United States
(12) Patent Application Publication
(10) Pub. No.: US 2025/0019354 A1
(43) Pub. Date: Jan. 16, 2025

(54) Crystal Modifications of Odevixibat

(71) Applicant: Albireo AB, Göteborg (SE)

(72) Inventors: Robert Lundqvist, Hälsö (SE); Ingvar Ymen, Saltsjo-boo (SE); Martin Bohlin, Johanneshov (SE); Eva Byröd, Mölndal (SE); Per-Göran Gillberg, Åsbro (SE); Anna-Maria Tivert, Göteborg (SE); Jessica Elversson, Dalby (SE); Nils Ove Gustafsson, Löddeköpinge (SE); Ann-Charlotte Dahlquist, Lund (SE); Rikard Bryland, Limhamn (SE)

(21) Appl. No.: 18/778,225

(22) Filed: Jul. 19, 2024

Related U.S. Application Data
(63) Continuation of application No. 17/744,429, filed on May 13, 2022, now Pat. No. 12,091,394.
Application No. 17/744,429 is a continuation of application No. 17/065,245, filed on Oct. 7, 2020, now Pat. No. 11,365,182.
Application No. 17/065,245 is a continuation of application No. 16/508,036, filed on Jul. 10, 2019, now Pat. No. 10,975,046.
Application No. 16/508,036 is a continuation of application No. PCT/SE2019/050602, filed on Jun. 20, 2019.

(30) Foreign Application Priority Data
Jun. 20, 2018 (SE) 1850761-6
Jun. 20, 2018 (SE) 1850762-4

(51) Int. Cl. C07D 285/36; A61K 9/10; A61K 9/48; A61K 9/50; A61K 31/554

(52) U.S. Cl. CPC C07D 285/36; A61K 9/10; A61K 9/4808; A61K 9/4816; A61K 9/4866; A61K 9/5042; A61K 9/5078; A61K 31/554; C07B 2200/13

(57) ABSTRACT

The present invention relates to crystal modifications of 1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine (odevixibat), more specifically crystal modifications 1 and 2 of odevixibat. The invention also relates to a process for the preparation of crystal modification 1 of odevixibat, to a pharmaceutical composition comprising crystal modification 1, and to the use of this crystal modification in the treatment of various conditions as described herein.

### CROSS-REFERENCE TO RELATED APPLICATIONS

This application is a continuation of U.S. patent application Ser. No. 17/744,429, filed on May 13, 2022, which is a continuation of U.S. patent application Ser. No. 17/065,245, filed Oct. 7, 2020, which is a continuation of U.S. patent application Ser. No. 16/508,036, filed Jul. 10, 2019, which is a continuation under 35 U.S.C. § 111(a) of International Application No. PCT/SE2019/050602, filed Jun. 20, 2019, which claims priority to Swedish Application No. 1850761-6, filed Jun. 20, 2018, and to Swedish Application No. 1850762-4, filed Jun. 20, 2018, the disclosures of which are incorporated by reference herein in their entireties.

### SEQUENCE LISTING

This application contains a Sequence Listing that has been submitted electronically as an XML file named “23854-0052004.XML.” The XML file, created on Jul. 19, 2024, is 14,705 bytes in size. The material in the XML file is hereby incorporated by reference in its entirety.

### TECHNICAL FIELD

The present invention relates to crystal modifications of 1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine (odevixibat), more specifically crystal modifications 1 and 2 of odevixibat. The invention also relates to a process for the preparation of crystal modification 1 of odevixibat, to a pharmaceutical composition comprising crystal modification 1, and to the use of this crystal modification in the treatment of various conditions as described herein.

### BACKGROUND

The compound 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat; also known as A4250) is disclosed in WO 03/022286. The structure of odevixibat is shown below.

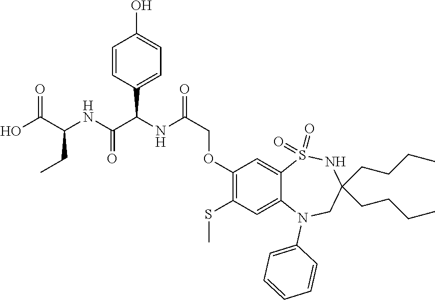 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

As an inhibitor of the ileal bile acid transporter (IBAT) mechanism, odevixibat inhibits the natural reabsorption of bile acids from the ileum into the hepatic portal circulation. Bile acids that are not reabsorbed from the ileum are instead excreted into the faeces. The overall removal of bile acids from the enterohepatic circulation leads to a decrease in the level of bile acids in serum and the liver. Odevixibat, or a pharmaceutically acceptable salt thereof, is therefore useful in the treatment or prevention of diseases such as dyslipidemia, constipation, diabetes and liver diseases, and especially liver diseases that are associated with elevated bile acid levels.

According to the experimental section of WO 03/022286, the last step in the preparation of odevixibat involves the hydrolysis of a tert-butyl ester under acidic conditions. The crude compound was obtained by evaporation of the solvent under reduced pressure followed by purification of the residue by preparative HPLC (Example 29). No crystalline material was identified.

Amorphous materials may contain high levels of residual solvents, which is highly undesirable for materials that should be used as pharmaceuticals. Also, because of their lower chemical and physical stability, as compared with crystalline material, amorphous materials may display faster decomposition and may spontaneously form crystals with a variable degree of crystallinity. This may result in unreproducible solubility rates and difficulties in storing and handling the material. In pharmaceutical preparations, the active pharmaceutical ingredient (API) is for that reason preferably used in a highly crystalline state. Thus, there is a need for crystal modifications of odevixibat having improved properties with respect to stability, bulk handling and solubility. In particular, it is an object of the present invention to provide a stable crystal modification of odevixibat that does not contain high levels of residual solvents, that has improved chemical stability and can be obtained in high levels of crystallinity.

### SUMMARY OF THE INVENTION

The invention provides crystal modifications of odevixibat. In a first aspect, the crystal modification is a crystalline hydrate of odevixibat. This crystalline hydrate is a channel hydrate, which may contain up to 2 moles of water associated with the crystal per mole of odevixibat. The amount of water calculated herein excludes water adsorbed to the surface of the crystal. In one embodiment, the crystalline hydrate is a sesquihydrate, i.e., contains about 1.5 moles of water associated with the crystal per mole of odevixibat. In another aspect, which may be related to the first aspect, the invention provides crystal modification 1 of odevixibat. Crystal modification 1 is a stable crystalline hydrate which at 30% relative humidity (RH) contains about 1.5 moles of water per mole of odevixibat.

In another aspect, the invention provides a dihydrate-disolvate of odevixibat. This mixed solvate can exist as different isostructural solvates and may comprise methanol, ethanol, 2-propanol, acetone, acetonitrile, 1,4-dioxane, DMF or DMSO as the organic solvent. When the mixed solvate is dried, it loses its solvate molecules and transforms into crystal modification 1 of odevixibat. In another aspect, which may be related to this aspect, the invention provides crystal modifications 2A, 2B and 2C of odevixibat, herein collectively referred to as crystal modification 2 of odevixibat. Upon drying, crystal modification 2 loses its organic solvent molecules and generates crystal modification 1 of odevixibat.

The invention further provides the use of crystal modification 1 of odevixibat in the treatment of a condition described herein, a pharmaceutical composition comprising crystal modification 1 of odevixibat, as well as a process for the preparation of crystal modification 1 of odevixibat.

### BRIEF DESCRIPTION OF THE DRAWINGS

FIG. 1 shows the X-ray powder diffractogram of dried crystal modification 1.

FIG. 2 shows the X-ray powder diffractogram of an overhydrated sample of crystal modification 1.

FIG. 3 shows the drying of crystal modification 1, with the X-ray powder diffractogram of an overhydrated sample of crystal modification 1 at the bottom and of a dried sample at the top (20 range 5-13°).

FIG. 4 shows the drying of crystal modification 1, with the X-ray powder diffractogram of an overhydrated sample of crystal modification 1 at the bottom and of a dry sample at the top (20 range 18-25°).

FIG. 5 shows the transformation from crystal modification 2 (bottom), as obtained from a mixture of ethanol (60-80% v/v) and water (20-40% v/v), to crystal modification 1 (top) via crystal modification 12 (middle).

FIG. 6 shows the X-ray powder diffractogram of crystal modification 2A, as obtained from a mixture of ethanol and water (70:30% v/v).

FIG. 7 shows the X-ray powder diffractogram of crystal modification 2A, as obtained from a mixture of acetone and water (50:50% v/v).

FIG. 8 shows the X-ray powder diffractogram of crystal modification 2A, as obtained from a mixture of 2-propanol and water (50:50% v/v).

FIG. 9 shows the X-ray powder diffractogram of crystal modification 2A, as obtained from a mixture 20 of 1,4-dioxane and water (50:50% v/v).

FIG. 10 shows the X-ray powder diffractogram of crystal modification 2B, as obtained from methanol. The water that is necessary for form 2 to crystallize was obtained from the air, as a result of the hygroscopicity of methanol.

FIG. 11 shows the X-ray powder diffractogram of crystal modification 2B, as obtained from a mixture of acetonitrile and water (40:60% v/v).

FIG. 12 shows the X-ray powder diffractogram of crystal modification 2C, as obtained from a mixture 30 of DMSO and water (50:50% v/v).

FIG. 13 shows the thermogravimetric analysis (TGA) mass change plot for crystal modification 1.

FIG. 14 shows the thermogravimetric analysis (TGA) mass change plot for crystal modification 2 produced by exposure of crystal modification 1 to the vapor phase of a mixture of ethanol and water

FIG. 15 shows the dynamic vapour sorption (DVS) mass change plot for crystal modification 1.

FIG. 16 shows the DSC trace of a sample of odevixibat with about 50% crystalline fraction (after pre-heating and cooling).

### DETAILED DESCRIPTION OF THE INVENTION

The invention described herein relates to crystal modifications that were discovered in extensive studies on odevixibat. It has been observed that odevixibat can crystallize from a variety of organic solvents (or mixtures of solvents) by incorporating solvate molecules in its structure, thereby forming various solvates or mixed solvates. While most of these (mixed) solvates are unstable in air and become amorphous upon drying, it has surprisingly been discovered that certain mixed solvates of odevixibat could be dried and transformed into a stable crystalline form of odevixibat. It is remarkable that this stable form, hereinafter referred to as crystal modification 1 of odevixibat, can be formed from different mixed solvates of odevixibat.

Thus, in a first aspect, the invention relates to crystal modification 1 of odevixibat. This stable crystal modification can be obtained from a slurry of odevixibat in a mixture of water and an organic solvent such as ethanol. Under these conditions, a mixed solvate containing about two moles of water and about one to about three, such as about two to about three, moles of ethanol per mole of odevixibat (e.g., a dihydrate-diethanolate or a dihydrate-triethanolate) is initially formed. In some embodiments, this mixed solvate is referred to as crystal modification 2. When the mixed solvate is dried, it loses its organic solvent molecules and becomes crystal modification 1. While not wishing to be bound by theory, it is believed that the solvent molecules can be removed without dissolution and recrystallization of the crystals.

Crystal modification 1 contains void volumes that are capable of containing up to about 2 moles of water associated with the crystal per mole of odevixibat, depending on the relative humidity. This form is therefore formally a channel hydrate. At about 30% relative humidity, however, crystal modification 1 contains a substantially stoichiometric amount of about 1.5 moles of water per mole of organic compound and is thus a sesquihydrate. The substantially stoichiometric amount of water is considered advantageous, as the water content of the crystals remains substantially constant even with humidity changes within the normal relative humidity range of about 30% to about 70% RH.

Indeed, at normal humidities, such as between about 30 and about 70% RH, crystal modification 1 exhibits relatively low hygroscopicity.

In one embodiment, the invention relates to crystal modification 1 of odevixibat having an X-ray powder diffraction (XRPD) pattern, obtained with CuKα1-radiation, with at least specific peaks at ° 2θ positions 5.6±0.2, 6.7±0.2 and/or 12.1±0.2.

In a specific embodiment thereof, the invention relates to crystal modification 1 having an XRPD pattern, obtained with CuKα1-radiation, with specific peaks at ° 2θ positions 5.6±0.2, 6.7±0.2 and 12.1±0.2 and one or more of the characteristic peaks: 4.1±0.2, 4.6±0.2, 9.3±0.2, 9.4±0.2 and 10.7±0.2.

In a more specific embodiment thereof, the invention relates to crystal modification 1 having an XRPD pattern, obtained with CuKα1-radiation, with specific peaks at ° 2θ positions 4.6±0.2, 5.6±0.2, 6.7±0.2, 9.3±0.2, 9.4±0.2 and 12.1±0.2.

In a yet more specific embodiment thereof, the invention relates to crystal modification 1 having an XRPD pattern, obtained with CuKα1-radiation, with characteristic peaks at ° 2θ positions 4.1±0.2, 4.6±0.2, 5.6±0.2, 6.7±0.2, 9.3±0.2, 9.4±0.2, 10.7±0.2 and 12.1±0.2, and one or more of 8.1±0.2, 8.6±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

In a yet even more specific embodiment thereof, the invention relates to crystal modification 1 having an XRPD pattern, obtained with CuKα1-radiation, with characteristic peaks at ° 2θ positions 4.1±0.2, 4.6±0.2, 5.6±0.2, 6.7±0.2, 8.1±0.2, 8.6±0.2, 9.3±0.2, 9.4±0.2, 10.7±0.2, 12.1±0.2, 13.4±0.2, 13.8±0.2, 13.9±0.2, 16.6±0.2, 17.3±0.2, 17.7±0.2, 18.3±0.2, 18.9±0.2, 19.4±0.2, 19.7±0.2, 20.5±0.2, 20.8±0.2, 21.6±0.2, 23.2±0.2, 24.3±0.2, 29.8±0.2 and 30.6±0.2.

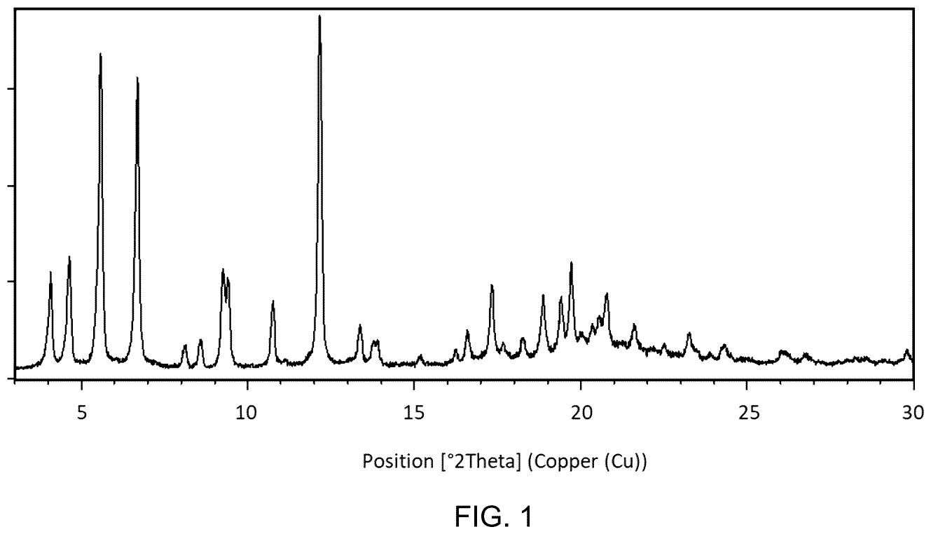

In a particular embodiment, the invention relates to crystal modification 1 having an XRPD pattern, obtained with CuKα1-radiation, substantially as shown in FIG. 1.

Whereas crystal modification 1 is a sesquihydrate containing about 3.5% (w/w) water at about 30% relative humidity (based on the total crystal weight), it has been observed that the crystal can take up an additional 1.5% (w/w) water when the humidity is increased up to 95% RH. The sorption and desorption of this additional water is fully reversible (see e.g. Example 10). The additional water may be adsorbed on the surface or may further fill the channels of the structure. In some embodiments, the term “overhydrated” refers to crystal modification 1 containing from about 1.5 to about 4 moles of water per mole of odevixibat, such as from about 1.5 to about 3.5, or such as from about 1.5 to 3, or such as from about 1.5 to about 2.5, or such as from about 1.5 to about 2 moles of water per mole of odevixibat. In some embodiments, the term “overhydrated” refers to crystal modification 1 containing from about 2 to about 4 moles of water per mole of odevixibat, such as from about 2 to about 3.5, or such as from about 2 to about 3, or such as from about 2 to 2.5 moles of water per mole of odevixibat.

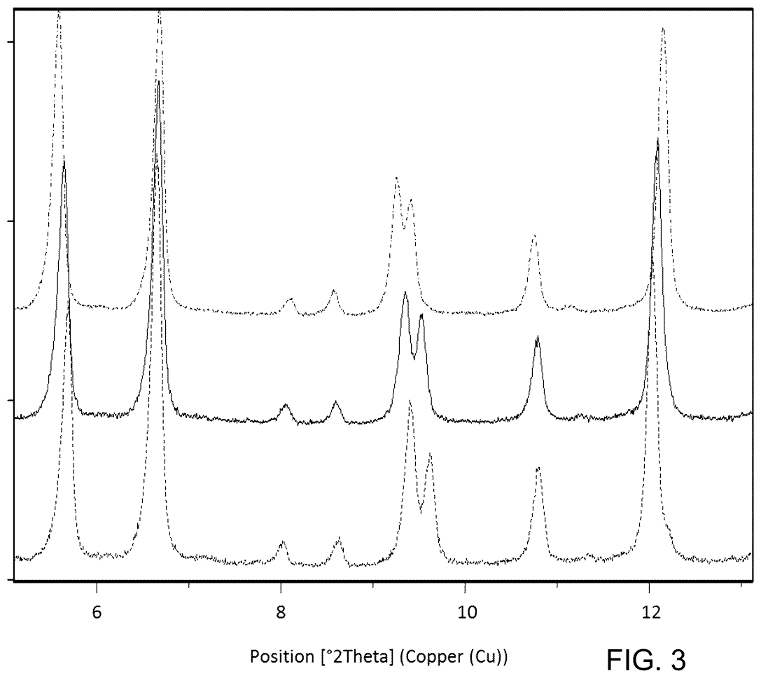

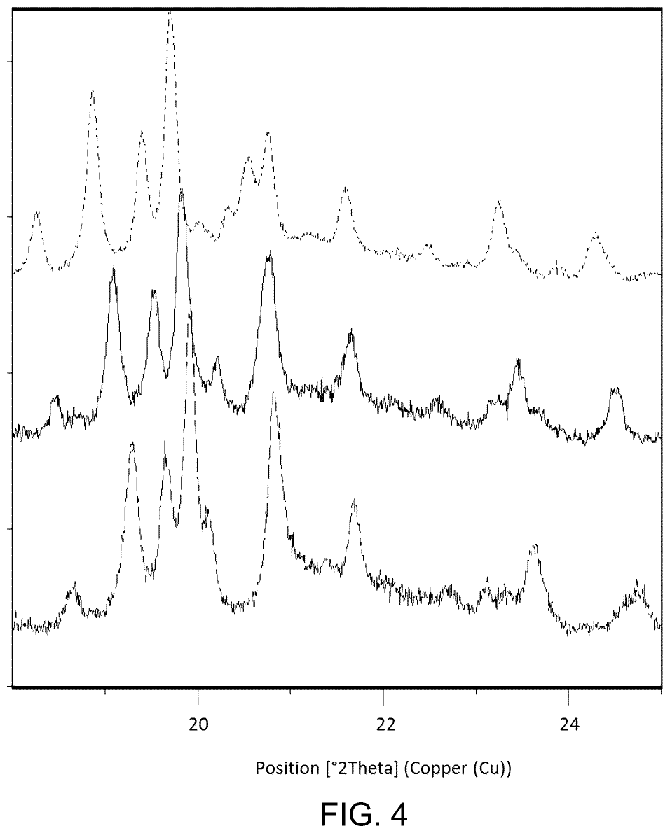

It has been observed that the XRPD pattern of overhydrated crystal modification 1 slightly changes when it is dried, e.g. at 50° C. in vacuum. A small shift of peaks is most clearly seen in the 2θ ranges 5 —13° and 18-25°, as shown in FIGS. 3 and 4, respectively. Exposing the dried modification to elevated relative humidity, such as up to 95% RH, makes the XRPD pattern of the overhydrated modification appear again. The peak shifts are a result of the unit cell volume changes, which occur as water molecules go in and out of the crystal structure.

Therefore, in another embodiment, the invention relates to overhydrated crystal modification 1 having an X-ray powder diffraction (XRPD) pattern, obtained with CuKα1-radiation, with at least specific peaks at ° 2θ positions 5.7±0.2, 6.7±0.2 and/or 12.0±0.2.

In certain embodiments, the invention relates to overhydrated crystal modification 1 having an XRPD pattern, obtained with CuKα1-radiation, with specific peaks at ° 2θ positions 5.7±0.2, 6.7±0.2 and 12.0±0.2 and one or more of the characteristic peaks: 4.0±0.2, 9.4±0.2, 9.6±0.2 and 10.8±0.2.

In a more particular embodiment, the invention relates to overhydrated crystal modification 1 having an XRPD pattern, obtained with CuKα1-radiation, with specific peaks at ° 2θ positions 4.0±0.2, 5.7±0.2, 6.7±0.2, 9.4±0.2, 9.6±0.2, 10.8±0.2 and 12.1±0.2.

In a further embodiment, the invention relates to overhydrated crystal modification 1 having an XRPD pattern, obtained with CuKα1-radiation, with characteristic peaks at ° 2θ positions 4.0±0.2, 5.7±0.2, 6.7±0.2, 9.4±0.2, 9.6±0.2, 10.8±0.2 and 12.1±0.2, and one or more of 4.7±0.2, 8.0±0.2, 8.6±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

In a yet further embodiment, the invention relates to overhydrated crystal modification 1 having an XRPD pattern, obtained with CuKα1-radiation, with characteristic peaks at ° 2θ positions 4.0±0.2, 4.7±0.2, 5.7±0.2, 6.7±0.2, 8.0±0.2, 8.6±0.2, 9.4±0.2, 9.6±0.2, 10.8±0.2, 12.1±0.2, 13.3±0.2, 14.1±0.2, 15.3±0.2, 16.5±0.2, 17.3±0.2, 19.3±0.2, 19.7±0.2, 19.9±0.2, 20.1±0.2, 20.8±0.2, 21.7±0.2, 23.6±0.2, 26.2±0.2, 26.5±0.2, 28.3±0.2 and 30.9±0.2.

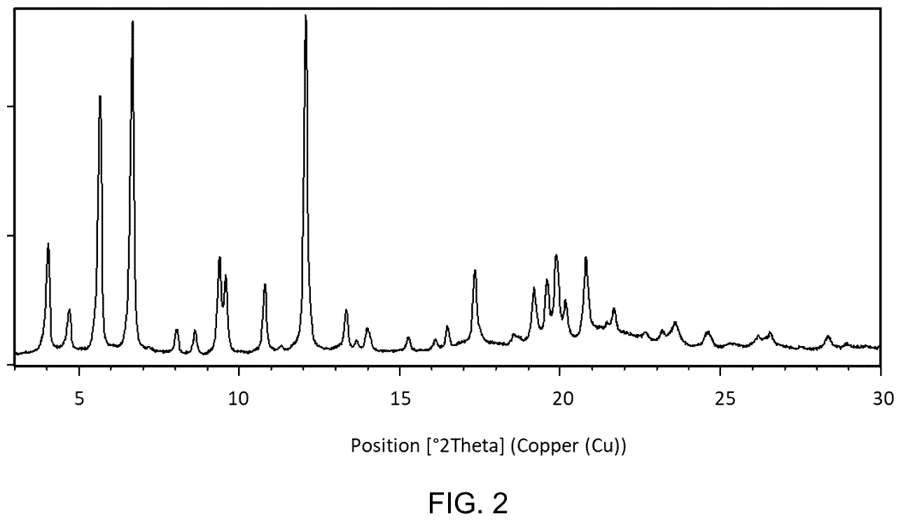

In yet another embodiment, the invention relates to overhydrated crystal modification 1 of odevixibat having an XRPD pattern, obtained with CuKα1-radiation, substantially as shown in FIG. 2.

In some embodiments, the crystallinity of crystal modification 1 is greater than about 99%. The crystallinity may be measured by Differential Scanning Calorimetry (DSC) methods, e.g. as disclosed in the experimental section.

Crystal modification 1 has several advantages over amorphous odevixibat. The relatively low hygroscopicity of crystal modification 1 at normal humidities, such as 30-70% RH, facilitates the handling and storing of odevixibat. Additionally, crystal modification 1 does not contain high levels of residual solvents. In contrast, it has been observed that batches of crude, amorphous odevixibat can contain residual solvents (such as formic acid) at levels that exceed the regulatory limits by far. Stability experiments have further shown that crystal modification 1 of odevixibat displays a higher chemical stability than amorphous odevixibat.

Crystal modification 1 may possess one or more additional advantages, such as a higher physical and thermodynamic stability than amorphous odevixibat; a more reproducible solubility than amorphous odevixibat; or an improved ability to process into a formulation. Such properties are highly relevant for pharmaceutical formulations of odevixibat.

In a second aspect, the invention relates to crystal modification 2 of odevixibat. It has been discovered that crystal modification 2 may be obtained not only from a mixture of ethanol and water, as described above, but also from methanol and certain other mixtures of solvent and water, including mixtures of methanol and water, 2-propanol and water, acetone and water, acetonitrile and water, 1,4-dioxane and water, DMF and water and DMSO and water. Crystal modification 2 is a mixed solvate, containing about two moles of water and about one to about three moles of organic solvent per mole of odevixibat. In some embodiments, the mixed solvate includes about 1.7 to about 2.3, about 1.8 to about 2.2, about 1.9 to about 2.1 or about 1.95 to about 2.05 moles of water associated with each mole of odevixibat in a crystal (excluding any water that may be adsorbed to the surface of the crystal).

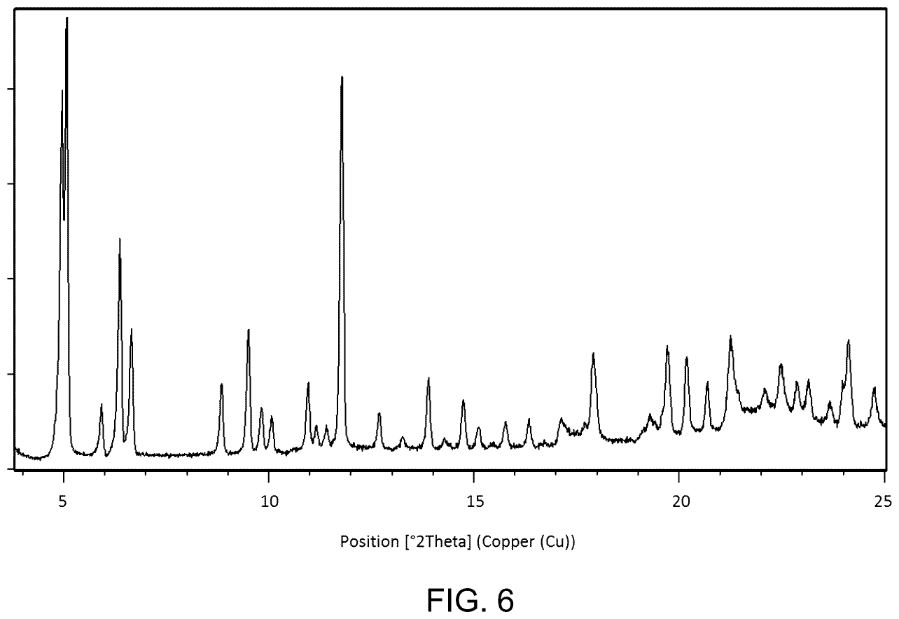

Interestingly, the XRPD patterns for the crystal modifications obtained from these different mixtures are essentially the same (see FIGS. 6-12). It is therefore believed that crystal modification 2 can exist as different isostructural solvates (also known as isomorphic solvates). In these isostructural solvates, crystal modification 2 accommodates different solvents (as a mixture with water). The presence of different solvents causes small volume changes to the unit cell but does not otherwise result in any significant distortion of the crystal structure of crystal modification 2. Nevertheless, the XRPD patterns for the isostructural solvates may be slightly different. Three similar, yet slightly different forms of crystal modification 2 are herein referred to as crystal modifications 2A, 2B and 2C, and collectively as “crystal modification 2”. Significantly, it has been found that upon drying, crystal modifications 2A, 2B and 2C can form crystal modification 1, regardless of the solvent mixture from which crystal modification 2 was crystallized.

In a first embodiment, the crystalline mixed solvate is crystal modification 2A, as obtained from a mixture of ethanol and water, acetone and water, 1,4-dioxane and water, DMF and water or 2-propanol and water, having an X-ray powder diffraction (XRPD) pattern, obtained with CuKα1-radiation, with at least specific peaks at ° 2θ positions 5.0±0.2, 5.1±0.2 and/or 11.8±0.2.

In a specific embodiment thereof, the invention relates to crystal modification 2A, as obtained from a mixture of ethanol and water, acetone and water, 1,4-dioxane and water, DMF and water or 2-propanol and water, having an XRPD pattern, obtained with CuKα1-radiation, with specific peaks at ° 2θ positions 5.0±0.2, 5.1±0.2 and 11.8±0.2 and one or more of the characteristic peaks: 6.4±0.2, 6.6±0.2 and 9.5±0.2.

In a more specific embodiment thereof, the invention relates to crystal modification 2A, as obtained from a mixture of ethanol and water, acetone and water, 1,4-dioxane and water, DMF and water or 2-propanol and water, having an XRPD pattern, obtained with CuKα1-radiation, with specific peaks at ° 2θ positions 5.0±0.2, 5.1±0.2, 6.4±0.2, 6.6±0.2, 9.5±0.2 and 11.8±0.2.

In a yet more specific embodiment thereof, the invention relates to crystal modification 2A, as obtained from a mixture of ethanol and water, having an XRPD pattern, obtained with CuKα1-radiation, with characteristic peaks at ° 20 5.0±0.2, 5.1±0.2, 6.4±0.2, 6.6±0.2, 9.5±0.2 and 11.8±0.2,and one or more of 5.9±0.2, 8.8±0.2, 9.8±0.2, 10.1±0.2, 11.0±0.2, 11.2±0.2, 11.4±0.2, 12.7±0.2, 13.9±0.2, 14.7±0.2, 15.1±0.2, 15.8±0.2, 16.3±0.2, 17.2±0.2, 17.9±0.2, 19.7±0.2, 20.2±0.2, 20.7±0.2, 21.3±0.2, 22.1±0.2, 22.5±0.2, 22.9±0.2, 23.2±0.2, 23.6±0.2, 24.0±0.2, 24.1±0.2, 24.7±0.2, 25.3±0.2, 26.7±0.2, 26.9±0.2, 29.8±0.2, 30.4±0.2, 30.8±0.2 and 31.6±0.2.

In a yet even more specific embodiment thereof, the invention relates to crystal modification 2A, as obtained from a mixture of ethanol and water, having an XRPD pattern, obtained with CuKα1-radiation, with characteristic peaks at ° 2θ positions 5.0±0.2, 5.1±0.2, 5.9±0.2, 6.4±0.2, 6.6±0.2, 8.8±0.2, 9.5±0.2, 9.8±0.2, 10.1±0.2, 11.0±0.2, 11.2±0.2, 11.4±0.2, 11.8±0.2, 12.7±0.2, 13.9±0.2, 14.7±0.2, 15.1±0.2, 15.8±0.2, 16.3±0.2, 17.2±0.2, 17.9±0.2, 19.7±0.2, 20.2±0.2, 20.7±0.2, 21.3±0.2, 22.1±0.2, 22.5±0.2, 22.9±0.2, 23.2±0.2, 23.6±0.2, 24.0±0.2, 24.1±0.2, 24.7±0.2, 25.3±0.2, 26.7±0.2, 26.9±0.2, 29.8±0.2, 30.4±0.2, 30.8±0.2 and 31.6±0.2.

In one particular embodiment, the invention relates to crystal modification 2A, as obtained from a mixture of ethanol and water, having an XRPD pattern, obtained with CuKα1-radiation, substantially as shown in FIG. 6.

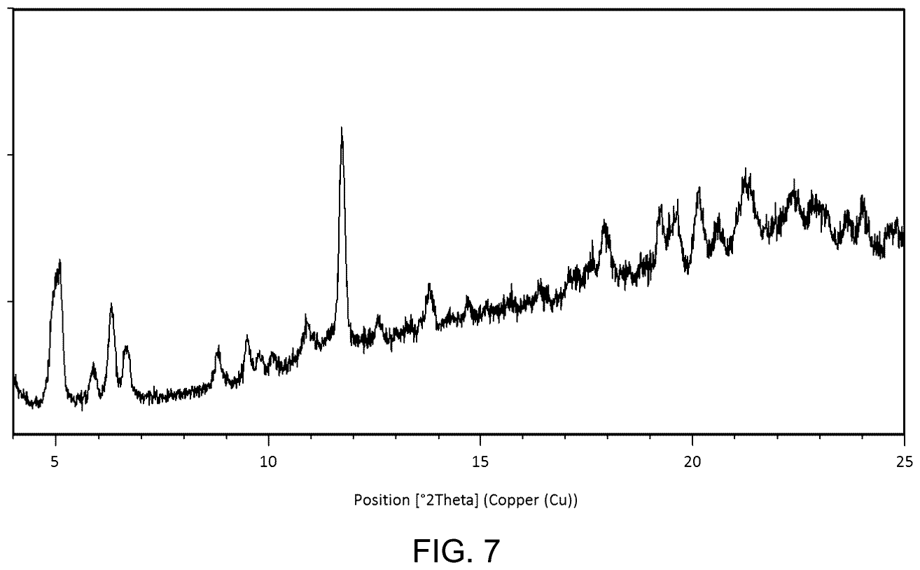

In another particular embodiment, the invention relates to crystal modification 2A, as obtained from a mixture of acetone and water, having an XRPD pattern, obtained with CuKα1-radiation, substantially as shown in FIG. 7.

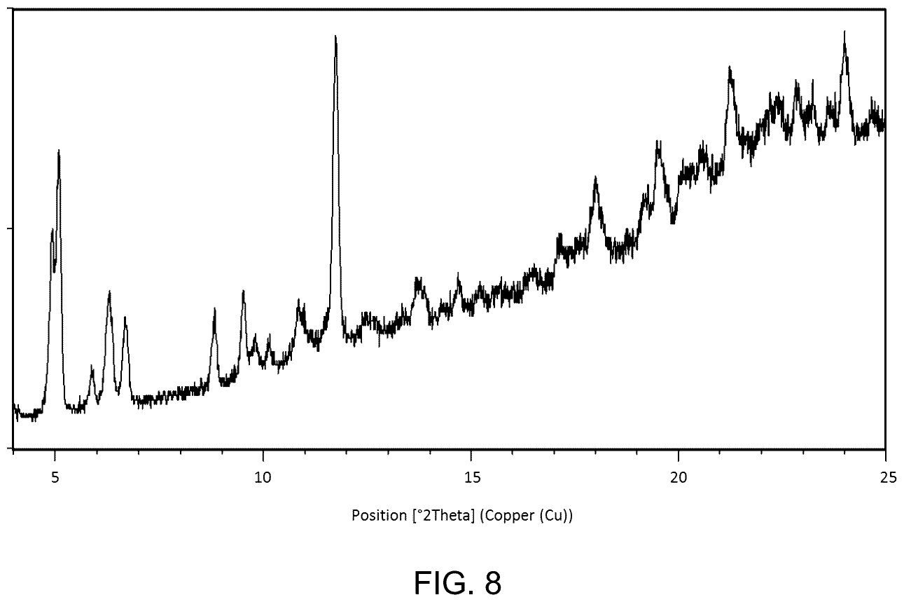

In yet another particular embodiment, the invention relates to crystal modification 2A, as obtained from a mixture of 2-propanol and water, having an XRPD pattern, obtained with CuKα1-radiation, substantially as shown in FIG. 8.

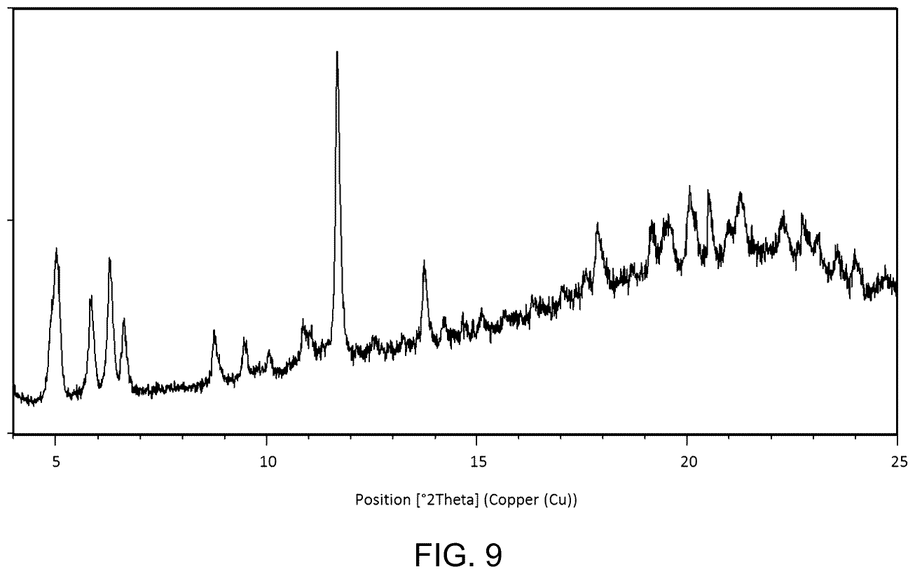

In yet another particular embodiment, the invention relates to crystal modification 2A, as obtained from a mixture of 1,4-dioxane and water, having an XRPD pattern, obtained with CuKα1-radiation, substantially as shown in FIG. 9.

In a second embodiment, the crystalline mixed solvate is crystal modification 2B, as obtained from methanol or from a mixture of methanol and water or acetonitrile and water, having an X-ray powder diffraction (XRPD) pattern, obtained with CuKα1-radiation, with at least specific peaks at ° 2θ positions 4.8±0.2, 5.1±0.2 and/or 11.6±0.2.

In a specific embodiment, the invention relates to crystal modification 2B, as obtained from methanol or from a mixture of methanol and water or acetonitrile and water, having an XRPD pattern, obtained with CuKα1-radiation, with specific peaks at ° 2θ positions 4.8±0.2, 5.1±0.2 and 11.6±0.2 and one or more of the characteristic peaks: 6.2±0.2, 6.7±0.2, 9.5±0.2 and 20.3±0.2.

In a more specific embodiment thereof, the invention relates to crystal modification 2B, as obtained from methanol or from a mixture of methanol and water or acetonitrile and water, having an XRPD pattern, obtained with CuKα1-radiation, with specific peaks at ° 2θ positions 4.8±0.2, 5.1±0.2, 6.2+±0.2, 67±0.2, 95±0.2, 11.6±0.2 and 20.3±0.2.

In a yet more specific embodiment thereof, the invention relates to crystal modification 2B, obtained from methanol and water, having an XRPD pattern, obtained with CuKα1-radiation, with characteristic peaks at ° 2θ positions 4.8±0.2, 5.1±0.2, 6.2±0.2, 6.7±0.2, 9.5±0.2, 11.6±0.2 and 20.3±0.2, and one or more of 5.8±0.2, 8.7±0.2, 9.7±0.2, 10.1±0.2, 10.7±0.2, 11.5±0.2, 13.4±0.2, 13.5±0.2, 14.4±0.2, 14.5±0.2, 15.2±0.2, 16.5±0.2, 16.8±0.2, 19.4±0.2, 20.6±0.2, 21.2±0.2, 21.5±0.2, 23.8±0.2, 23.9±0.2, 25.4±0.2, 26.3±0.2, 26.7±0.2, 30.1±0.2 and 30.6±0.2.

In a yet even more specific embodiment thereof, the invention relates to crystal modification 2B, obtained from methanol and water, having an XRPD pattern, obtained with CuKα1-radiation, with characteristic peaks at ° 2θ positions 4.8±0.2, 5.1±0.2, 5.8±0.2, 6.2±0.2, 6.7±0.2, 8.7±0.2, 9.5±0.2, 9.7±0.2, 10.1±0.2, 10.7±0.2, 11.5±0.2, 11.6±0.2, 13.4±0.2, 13.5±0.2, 14.4±0.2, 14.5±0.2, 15.2±0.2, 16.5±0.2, 16.8±0.2, 19.4±0.2, 20.3±0.2, 20.6±0.2, 21.2±0.2, 21.5±0.2, 23.8±0.2, 23.9±0.2, 25.4±0.2, 26.3±0.2, 26.7±0.2, 30.1±0.2 and 30.6±0.2.

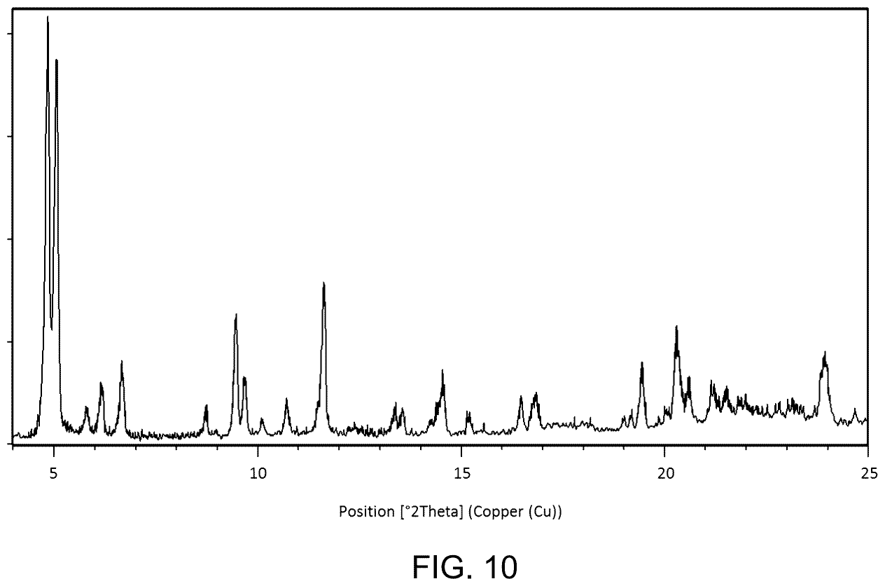

In one particular embodiment, the invention relates to crystal modification 2B, as obtained from methanol, having an XRPD pattern, obtained with CuKα1-radiation, substantially as shown in FIG. 10.

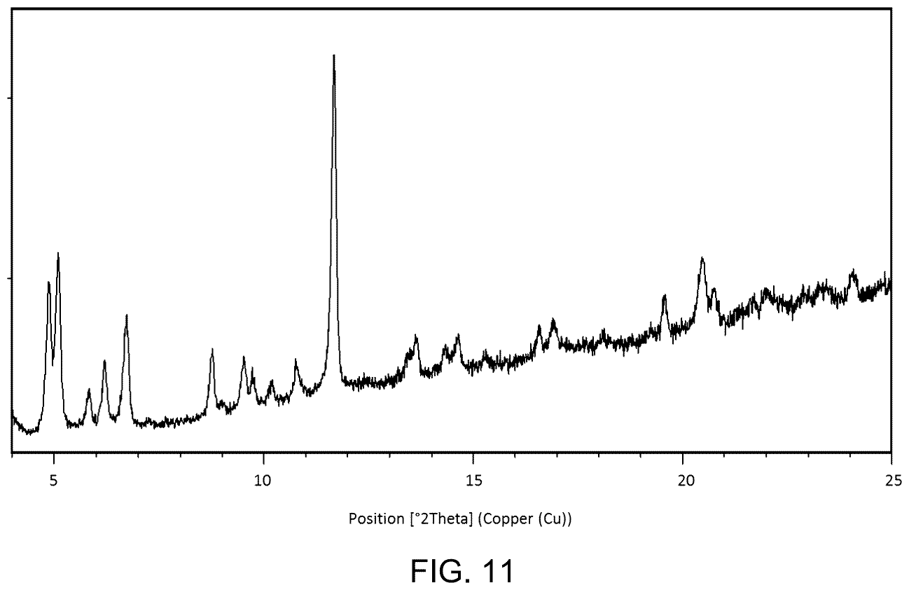

In another particular embodiment, the invention relates to crystal modification 2B, as obtained from a mixture of acetonitrile and water, having an XRPD pattern, obtained with CuKα1-radiation, substantially as shown in FIG. 11.

In a third embodiment, the invention relates to crystal modification 2C, as obtained from a mixture of DMSO and water, having an X-ray powder diffraction (XRPD) pattern, obtained with CuKα1-radiation, with at least specific peaks at ° 2θ positions 5.0±0.2, 6.2±0.2, 9.4±0.2 and/or 23.9±0.2.

In a specific embodiment thereof, the invention relates to crystal modification 2C, as obtained from a mixture of DMSO and water, having an XRPD pattern, obtained with CuKα1-radiation, with specific peaks at ° 2θ positions 5.0±0.2, 6.2±0.2, 9.4±0.2 and 23.9±0.2 and one or more of the characteristic peaks: 11.5±0.2, 19.5±0.2 and 20.2±0.2.

In a more specific embodiment thereof, the invention relates to crystal modification 2C, as obtained from a mixture of DMSO and water, having an XRPD pattern, obtained with CuKα1-radiation, with specific peaks at ° 2θ positions 5.0±0.2, 6.2±0.2, 9.4±0.2, 11.5±0.2, 19.5±0.2, 20.2 0.2 and 23.9±0.2.

In a yet more specific embodiment thereof, the invention relates to crystal modification 2C, as obtained from a mixture of DMSO and water, having an XRPD pattern, obtained with CuKα1-radiation, with characteristic peaks at ° 2θ positions 5.0±0.2, 6.2±0.2, 9.4±0.2, 11.5±0.2, 19.5±0.2, 20.2±0.2 and 23.9±0.2, and one or more of 4.9±0.2, 5.8±0.2, 6.6±0.2, 8.6±0.2, 9.7±0.2, 10.0±0.2, 10.8±0.2, 13.5±0.2, 15.1±0.2, 17.7±0.2, 17.9±0.2, 19.0±0.2, 19.3±0.2, 20.7±0.2, 21.1±0.2, 21.2±0.2, 21.2±0.2, 22.8±0.2, 25.3±0.2, 26.6±0.2, 27.3±0.2, 27.4±0.2, 28.6±0.2, 30.1±0.2 and 30.2±0.2.

In a yet even more specific embodiment thereof, the invention relates to crystal modification 2C, as obtained from a mixture of DMSO and water, having an XRPD pattern, obtained with CuKα1-radiation, with characteristic peaks at ° 2θ positions 4.9±0.2, 5.0±0.2, 5.8±0.2, 6.2±0.2, 6.6±0.2, 8.6±0.2, 9.4±0.2, 9.7±0.2, 10.0±0.2, 10.8±0.2, 11.5±0.2, 13.5±0.2, 15.1±0.2, 17.7±0.2, 17.9±0.2, 19.0±0.2, 19.3±0.2, 19.5±0.2, 20.2±0.2, 20.7±0.2, 21.1±0.2, 21.2±0.2, 21.3±0.2, 22.8±0.2, 23.9±0.2, 25.3±0.2, 26.6±0.2, 27.3±0.2, 27.4±0.2, 28.6±0.2, 30.1±0.2 and 30.2±0.2.

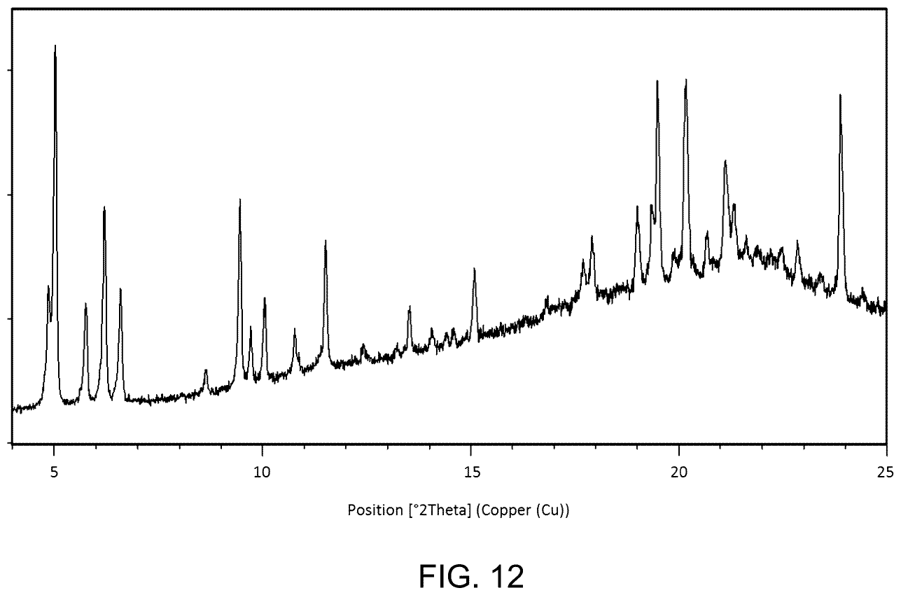

In one particular embodiment, the invention relates to crystal modification 2C, as obtained from a mixture of DMSO and water, having an XRPD pattern, obtained with CuKα1-radiation, substantially as shown in FIG. 12.

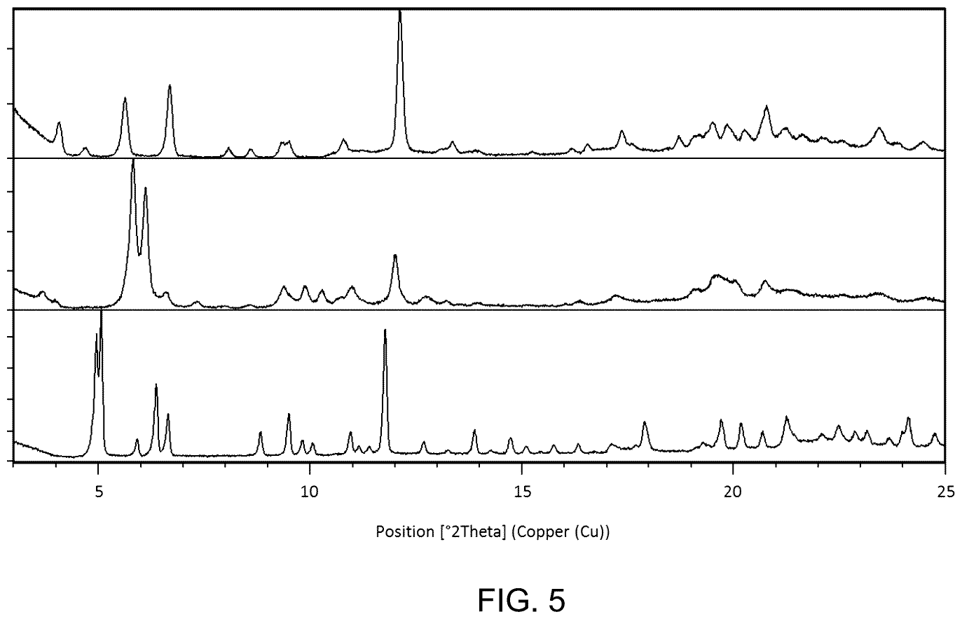

As will be understood from the above, the isolation and characterization of stable crystal modification 1 was not straightforward. Even though it is a hydrate, crystal modification 1 cannot be obtained directly by crystallization from water. In some embodiments, crystal modification 1 is obtained indirectly, e.g. by isolating and drying crystal modification 2, which is formed by crystallization of odevixibat from mixtures of water and certain organic solvents. In some embodiments, crystal modification 1 is obtained from crystal modification 2 after evaporation of the solvent molecules. In some embodiments, the transformation of crystal modification 2 to crystal modification 1 proceeds via a crystalline intermediate, namely modification 12 (see FIG. 5). In some embodiments, the solvent molecules are removed from modification 2 without dissolution and recrystallization of the crystals.

In another aspect, the invention relates to the use of crystal modification 2 (2A, 2B or 2C) of odevixibat as described herein in a process for the preparation of crystal modification 1 of odevixibat.

In yet another aspect, the invention relates to a process for the preparation of crystal modification 1 of odevixibat. In some embodiments, this process involves isolating crystal modification 2 of odevixibat from a solution of odevixibat in a solvent mixture comprising water and an organic solvent selected from the group consisting of methanol, ethanol, 2-propanol, acetone, acetonitrile, 1,4-dioxane, DMF and DMSO, and mixtures thereof. In some embodiments, the process involves isolating crystal modification 2 of odevixibat from a solution of odevixibat in a solvent mixture comprising water and an organic solvent selected from the group consisting of methanol, ethanol, 2-propanol, acetone, acetonitrile, 1,4-dioxane, DMF and DMSO.

In some embodiments, the crystallinity of crystal modification 1 is dependent on the drying process.

As is shown in the experimental section, it has been observed that superior crystallinity of crystal modification 1 can be obtained when crystal modification 2 is dried under vacuum (e.g., less than 5 mbar) or under a nitrogen flow. It is believed that drying of crystal modification 2 under these conditions results in a dehydrated form, which then quickly takes up water from the air.

In some embodiments, therefore, the process for the preparation of crystal modification 1 of odevixibat comprises the steps of:
a) isolating crystal modification 2 of odevixibat from a solution of odevixibat in a solvent mixture comprising water and an organic solvent selected from the group consisting of methanol, ethanol, 2-propanol, acetone, acetonitrile, 1,4-dioxane, DMF and DMSO; and
b) drying the solid under vacuum or under a nitrogen flow.

In a preferred embodiment, crystal modification 2 of odevixibat is crystal modification 2A of odevixibat. In a more preferred embodiment, crystal modification 2A of odevixibat is obtained from a mixture of water and ethanol.

In some embodiments, the process for the preparation of crystal modification 1 of odevixibat comprises the steps of:
a) isolating crystal modification 2A of odevixibat from a solution of odevixibat in a mixture of water and ethanol; and
b) drying the solid under vacuum or under a nitrogen flow.

In some embodiments, the crystallinity of crystal modification 1 is dependent on the composition of the mixture of water and the organic solvent. For example, superior crystallinity of crystal modification 1 can be obtained from samples of crystal modification 2A that are obtained from a slurry of odevixibat in a 60:40 (% v/v) mixture of ethanol and water at 22° C. In a preferred embodiment, the ethanol content in the solvent mixture is about 55 to about 75% (v/v), such as about 60 to about 70% (v/v). In some embodiments, the ethanol content in the solvent mixture is about 60% (v/v). In some embodiments, the ethanol content in the solvent mixture is about 65% (v/v). In some embodiments, the ethanol content in the solvent mixture is about 70% (v/v).

In some embodiments, the crystallinity of crystal modification 2A is increased when the isolated crystals are exposed to an ethanol/water atmosphere containing 40 to 60% (v/v) ethanol for a period of at least 24 hours.

In some embodiments, the process comprises the steps of:
a) preparing a saturated solution of odevixibat in a mixture of water and an organic solvent selected from the group consisting of methanol, ethanol, 2-propanol, acetone, acetonitrile, 1,4-dioxane, DMF and DMSO;
b) adding an excess of odevixibat to the saturated solution of step a) so as to obtain a slurry;
c) maintaining stirring of the slurry at a temperature of about 0 to about 25° C., for a period of at least 24 hours;
d) recovering the solid obtained in step c);
e) drying the solid under vacuum or under a nitrogen flow.

In some embodiments, the process comprises the steps of:
a) preparing a saturated solution of odevixibat in a mixture of water and ethanol;
b) adding an excess of odevixibat to the saturated solution of step a) so as to obtain a slurry;
c) maintaining stirring of the slurry at a temperature of about 20 to about 25° C., preferably about 22° C., for a period of at least 24 hours;
d) recovering the solid obtained in step c);
e) optionally exposing the crystals of step d) to an ethanol/water atmosphere; and
f) drying the solid under vacuum or under a nitrogen flow.

Alternatively, crystal modification 1 can be obtained by adding seed crystals to a saturated solution of odevixibat in a mixture of water and a suitable organic solvent. Thus, in another embodiment, the process comprises the steps of:
a) preparing a saturated solution of odevixibat in a mixture of water and an organic solvent selected from the group consisting of methanol, ethanol, 2-propanol, acetone, acetonitrile, 1,4-dioxane, DMF and DMSO;
b) adding seed crystals to the saturated solution of step a);
c) maintaining stirring of the slurry at a temperature of about 0 to about 25° C., for a period of at least 24 hours;
d) recovering the solid obtained in step c);
e) drying the solid under vacuum or under a nitrogen flow.

In some embodiments, the process comprises the steps of:
a) preparing a saturated solution of odevixibat in a mixture of water and ethanol;
b) adding seed crystals to the saturated solution of step a);
c) maintaining stirring of the slurry at a temperature of about 20 to about 25° C., preferably 22° C., for a period of at least 24 hours;
d) recovering the solid obtained in step c);
e) optionally exposing the crystals of step d) to an ethanol/water atmosphere; and
f) drying the solid under vacuum or under a nitrogen flow.

A slurry sample of crystal modification 2 may be used as the seed crystals. Alternatively, crystal modification 1 may be used. It is believed that this form quickly transforms into crystal modification 2 when added to the solvent mixture of the crystallization process.

In a further aspect, the invention relates to crystalline modification 1 of odevixibat, prepared by a process comprising the steps of:
a) isolating crystal modification 2 of odevixibat from a solution of odevixibat in a solvent mixture comprising water and an organic solvent selected from the group consisting of methanol, ethanol, 2-propanol, acetone, acetonitrile, 1,4-dioxane, DMF and DMSO; and
b) drying the solid under vacuum or under a nitrogen flow.

In a further aspect, the invention also relates to crystal modification 1 of odevixibat as described herein for use in therapy.

Odevixibat is an ileal bile acid transporter (IBAT) inhibitor. The ileal bile acid transporter (IBAT) is the main mechanism for re-absorption of bile acids from the GI tract. Partial or full blockade of that odevixibat mechanism will result in lower concentration of bile acids in the small bowel wall, portal vein, liver parenchyma, intrahepatic biliary tree, and extrahepatic biliary tree, including the gall bladder. Diseases which may benefit from partial or full blockade of the IBAT mechanism may be those having, as a primary pathophysiological defect, symptoms of excessive concentration of bile acids in serum and in the above organs. Crystal modification 1 of odevixibat, as described herein, is therefore useful in the treatment or prevention of conditions, disorders and diseases wherein inhibition of the bile acid circulation is desirable, such as cardiovascular diseases, fatty acid metabolism and glucose utilization disorders, gastrointestinal diseases and disorders, liver diseases and disorders.

Cardiovascular diseases and disorders of fatty acid metabolism and glucose utilization include, but are not limited to, hypercholesterolemia; disorders of fatty acid metabolism; type 1 and type 2 diabetes mellitus; complications of diabetes, including cataracts, micro- and macrovascular diseases, retinopathy, neuropathy, nephropathy and delayed wound healing, tissue ischaemia, diabetic foot, arteriosclerosis, myocardial infarction, acute coronary syndrome, unstable angina pectoris, stable angina pectoris, stroke, peripheral arterial occlusive disease, cardiomyopathy, heart failure, heart rhythm disorders and vascular restenosis; diabetes-related diseases such as insulin resistance (impaired glucose homeostasis), hyperglycemia, hyperinsulinemia, elevated blood levels of fatty acids or glycerol, obesity, dyslipidemia, hyperlipidemia including hypertriglyceridemia, metabolic syndrome (syndrome X), atherosclerosis and hypertension; and for increasing high density lipoprotein levels.

Gastrointestinal diseases and disorders include constipation (including chronic constipation, functional constipation, chronic idiopathic constipation (CIC), intermittent/sporadic constipation, constipation secondary to diabetes mellitus, constipation secondary to stroke, constipation secondary to chronic kidney disease, constipation secondary to multiple sclerosis, constipation secondary to Parkinson's disease, constipation secondary to systemic sclerosis, drug induced constipation, irritable bowel syndrome with constipation (IBS-C), irritable bowel syndrome mixed (IBS-M), pediatric functional constipation and opioid induced constipation); Crohn's disease; primary bile acid malabsorption; irritable bowel syndrome (IBS); inflammatory bowel disease (IBD); ileal inflammation; and reflux disease and complications thereof, such as Barrett's esophagus, bile reflux esophagitis and bile reflux gastritis. The treatment and prevention of constipation has also been disclosed in WO 2004/089350, which is incorporated by reference in its entirety herein.

A liver disease as defined herein is any disease in the liver and in organs connected therewith, such as the pancreas, portal vein, the liver parenchyma, the intrahepatic biliary tree, the extrahepatic biliary tree, and the gall bladder. In some embodiments, a liver disease a bile acid-dependent liver disease. In some embodiments, a liver disease involves elevated levels of bile acids in the serum and/or in the liver. In some embodiments, a liver disease is a cholestatic liver disease. Liver diseases and disorders include, but are not limited to an inherited metabolic disorder of the liver; inborn errors of bile acid synthesis; congenital bile duct anomalies; biliary atresia; post-Kasai biliary atresia; post-liver transplantation biliary atresia; neonatal hepatitis; neonatal cholestasis; hereditary forms of cholestasis; cerebrotendinous xanthomatosis; a secondary defect of BA synthesis; Zellweger's syndrome; cystic fibrosis-associated liver disease; alpha1-antitrypsin deficiency; Alagilles syndrome (ALGS); Byler syndrome; a primary defect of bile acid (BA) synthesis; progressive familial intrahepatic cholestasis (PFIC) including PFIC-1, PFIC-2, PFIC-3 and non-specified PFIC, post-biliary diversion PFIC and post-liver transplant PFIC; benign recurrent intrahepatic cholestasis (BRIC) including BRIC1, BRIC2 and non-specified BRIC, post-biliary diversion BRIC and post-liver transplant BRIC; autoimmune hepatitis; primary biliary cirrhosis (PBC); liver fibrosis; non-alcoholic fatty liver disease (NAFLD); non-alcoholic steatohepatitis (NASH); portal hypertension; cholestasis; Down syndrome cholestasis; drug-induced cholestasis; intrahepatic cholestasis of pregnancy (jaundice during pregnancy); intrahepatic cholestasis; extrahepatic cholestasis; parenteral nutrition associated cholestasis (PNAC); low phospholipid-associated cholestasis; lymphedema cholestasis syndrome 1 (LSC1); primary sclerosing cholangitis (PSC); immunoglobulin G4 associated cholangitis; primary biliary cholangitis; cholelithiasis (gall stones); biliary lithiasis; choledocholithiasis; gallstone pancreatitis; Caroli disease; malignancy of bile ducts; malignancy causing obstruction of the biliary tree; biliary strictures; AIDS cholangiopathy; ischemic cholangiopathy; pruritus due to cholestasis or jaundice; pancreatitis; chronic autoimmune liver disease leading to progressive cholestasis; hepatic steatosis; alcoholic hepatitis; acute fatty liver; fatty liver of pregnancy; drug-induced hepatitis; iron overload disorders; congenital bile acid synthesis defect type 1 (BAS type 1); drug-induced liver injury (DILI); hepatic fibrosis; congenital hepatic fibrosis; hepatic cirrhosis; Langerhans cell histiocytosis (LCH); neonatal ichthyosis sclerosing cholangitis (NISCH); erythropoietic protoporphyria (EPP); idiopathic adulthood ductopenia (IAD); idiopathic neonatal hepatitis (INH); non syndromic paucity of interlobular bile ducts (NS PILBD); North American Indian childhood cirrhosis (NAIC); hepatic sarcoidosis; amyloidosis; necrotizing enterocolitis; serum bile acid-caused toxicities, including cardiac rhythm disturbances (e.g., atrial fibrillation) in setting of abnormal serum bile acid profile, cardiomyopathy associated with liver cirrhosis (“cholecardia”), and skeletal muscle wasting associated with cholestatic liver disease; viral hepatitis (including hepatitis A, hepatitis B, hepatitis C, hepatitis D and hepatitis E); hepatocellular carcinoma (hepatoma); cholangiocarcinoma; bile acid-related gastrointestinal cancers; and cholestasis caused by tumours and neoplasms of the liver, of the biliary tract and of the pancreas. The treatment and prevention of liver diseases has also been disclosed in WO 2012/064266, which is incorporated by reference in its entirety herein.

Other diseases that may be treated or prevented by crystal modification 1 of odevixibat include hyperabsorption syndromes (including abetalipoproteinemia, familial hypobetalipoproteinemia (FHBL), chylomicron retention disease (CRD) and sitosterolemia); hypervitaminosis and osteopetrosis; hypertension; glomerular hyperfiltration; and pruritus of renal failure.

Biliary atresia is a rare pediatric liver disease that involves a partial or total blockage (or even absence) of large bile ducts. This blockage or absence causes cholestasis that leads to the accumulation of bile acids that damages the liver. In some embodiments, the accumulation of bile acids occurs in the extrahepatic biliary tree. In some embodiments, the accumulation of bile acids occurs in the intrahepatic biliary tree. The current standard of care is the Kasai procedure, which is a surgery that removes the blocked bile ducts and directly connects a portion of the small intestine to the liver. There are currently no approved drug therapies for this disorder.

Provided herein are methods for treating biliary atresia in a subject in need thereof, the methods comprising administration of a therapeutically effective amount of crystal modification 1 of odevixibat. In some embodiments, the subject has undergone the Kasai procedure prior to administration of a crystal modification 1 of odevixibat. In some embodiments, the subject is administered crystal modification 1 of odevixibat prior to undergoing the Kasai procedure. In some embodiments, the treatment of biliary atresia decreases the level of serum bile acids in the subject. In some embodiments, the level of serum bile acids is determined by, for example, an ELISA enzymatic assay or the assays for the measurement of total bile acids as described in Danese et al., PLoS One. 2017, vol. 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of crystal modification 1 of odevixibat. In some embodiments, the treatment of biliary atresia includes treatment of pruritus.

PFIC is a rare genetic disorder that is estimated to affect between one in every 50,000 to 100,000 children born worldwide and causes progressive, life-threatening liver disease.

One manifestation of PFIC is pruritus, which often results in a severely diminished quality of life. In some cases, PFIC leads to cirrhosis and liver failure. Current therapies include Partial External Biliary Diversion (PEBD) and liver transplantation, however, these options can carry substantial risk of post-surgical complications, as well as psychological and social issues.

Three alternative gene defects have been identified that correlate to three separate PFIC subtypes known as types 1, 2 and 3.
PFIC, type 1, which is sometimes referred to as “Byler disease,” is caused by impaired bile secretion due to mutations in the ATP8B1 gene, which codes for a protein that helps to maintain an appropriate balance of fats known as phospholipids in cell membranes in the bile ducts. An imbalance in these phospholipids is associated with cholestasis and elevated bile acids in the liver. Subjects affected by PFIC, type 1 usually develop cholestasis in the first months of life and, in the absence of surgical treatment, progress to cirrhosis and end-stage liver disease before the end of the first decade of life.
PFIC, type 2, which is sometimes referred to as “Byler syndrome,” is caused by impaired bile salt secretion due to mutations in the ABCB11 gene, which codes for a protein, known as the bile salt export pump, that moves bile acids out of the liver. Subjects with PFIC, type 2 often develop liver failure within the first few years of life and are at increased risk of developing a type of liver cancer known as hepatocellular carcinoma.
PFIC, type 3, which typically presents in the first years of childhood with progressive cholestasis, is caused by mutations in the ABCB4 gene, which codes for a transporter that moves phospholipids across cell membranes.

In addition, TJP2 gene, NR1H4 gene or Myo5b gene mutations have been proposed to be causes of PFIC. In addition, some subjects with PFIC do not have a mutation in any of the ATP8B1, ABCB11, ABCB4, TJP2, NR1H4 or Myo5b genes. In these cases, the cause of the condition is unknown.

Exemplary mutations of the ATP8B1 gene or the resulting protein are listed in Tables 1 and 2, with numbering based on the human wild type ATP8B1 protein (e.g., SEQ ID NO: 1) or gene (e.g., SEQ ID NO: 2). Exemplary mutations of the ABCB11 gene or the resulting protein are listed in Tables 3 and 4, with numbering based on the human wild type ABCB11 protein (e.g., SEQ ID NO: 3) or gene (e.g., SEQ ID NO: 4).

As can be appreciated by those skilled in the art, an amino acid position in a reference protein sequence that corresponds to a specific amino acid position in SEQ ID NO: 1 or 3 can be determined by aligning the reference protein sequence with SEQ ID NO: 1 or 3 (e.g., using a software program, such as ClustalW2). Changes to these residues (referred to herein as “mutations”) may include single or multiple amino acid substitutions, insertions within or flanking the sequences, and deletions within or flanking the sequences. As can be appreciated by those skilled in the art, an nucleotide position in a reference gene sequence that corresponds to a specific nucleotide position in SEQ ID NO: 2 or 4 can be determined by aligning the reference gene sequence with SEQ ID NO: 2 or 4 (e.g., using a software program, such as ClustalW2). Changes to these residues (referred to herein as “mutations”) may include single or multiple nucleotide substitutions, insertions within or flanking the sequences, and deletions within or flanking the sequences. See also Kooistra, et al., “KLIFS: A structural kinase-ligand interaction database,” Nucleic Acids Res. 2016, vol. 44, no. D, pp. D365-D371, which is incorporated by reference in its entirety herein.

TABLE 1
Exemplary ATP8B1 Mutations

Amino acid position 3 (e.g., T3K)²⁷
Amino acid position 23 (e.g., P23L)⁵
Amino acid position 45 (e.g., N45T)^{5,8,9}
Amino acid position 46 (e.g., R46X)^{A,25}
Amino acid position 62 (e.g., C62R)²⁸
Amino acid position 63 (e.g., T63T)⁴¹
Amino acid position 70 (e.g., D70N)^{1,6}
Amino acid position 71 (e.g., R71H)⁴³
Amino acid position 78 (e.g., H78Q)¹⁹
Amino acid position 82 (e.g., T82T)⁴¹
Amino acid position 92 (e.g., Y92Y)⁴¹
Amino acid position 93 (e.g., A93A)⁶
Amino acid position 96 (e.g., A96G)²⁷
Amino acid position 114 (e.g., E114Q)⁸
Amino acid position 127 (e.g., L127P⁶, L127V³⁶)
Amino acid position 177 (e.g., T177T)⁶
Amino acid position 179 (e.g., E179X)²⁹
Δ Amino acid positions 185-282⁴⁴
Amino acid position 197 (e.g., G197Lfs*10)²²
Amino acid position 201 (e.g., R201S²⁷, R201H³⁵)
Amino acid position 203 (e.g., K203E^{5,8}, K203R⁹, K203fs²⁵)
Amino acid position 205 (e.g., N205fs⁶, N205Kfs*2³⁵)
Amino acid position 209 (e.g., P209T)⁴
Amino acid position 217 (e.g., S217N)⁴³
Amino acid position 232 (e.g., D232D)³⁰
Amino acid position 233 (e.g., G233R)³⁸
Amino acid position 243 (e.g., L243fs*28)³³
Amino acid position 265 (e.g., C265R)²⁵
Amino acid position 271 (e.g., R271X¹³, R271R³⁰)
Amino acid position 288 (e.g., L288S)⁶
Amino acid position 294 (e.g., L294S)⁴³
Amino acid position 296 (e.g., R296C)¹¹
Amino acid position 305 (e.g., F305I)²⁸
Amino acid position 306 (e.g., C306R)²³
Amino acid position 307 (e.g., H307L)³⁵
Amino acid position 308 (e.g., G308V¹, G308D⁶, G308S³⁵)
Amino acid position 314 (e.g., G314S)¹³
Amino acid position 320 (e.g., M320Vfs*13)¹¹
Amino acid position 337 (e.g., M337R)¹⁸
Amino acid position 338 (e.g., N338K)¹⁸
Amino acid position 340 (e.g., M340V)¹⁸
Amino acid position 344 (e.g., I344F)^{6,20}
Amino acid position 349 (e.g., I349T)⁴¹
Amino acid position 358 (e.g., G358R)²⁸
Amino acid position 367 (e.g., G367G)⁴¹
Amino acid position 368 (e.g., N368D)⁴¹
Amino acid position 393 (e.g., I393V)²⁷
Amino acid position 403 (e.g., S403Y)⁶
Amino acid position 407 (e.g., S407N)⁴⁰
Amino acid position 412 (e.g., R412P)⁶
Amino acid position 415 (e.g., Q415R)²⁷
Amino acid position 422 (e.g., D422H)³⁵
Amino acid position 429 (e.g., E429A)⁶
Amino acid position 446 (e.g., G446R)^{4,11}
Amino acid position 453 (e.g., S453Y)⁶
Amino acid position 454 (e.g., D454G)⁶
Amino acid position 455 (e.g., K455N)⁴³
Amino acid position 456 (e.g., T456M^{3,6}, T456K³⁵)
Amino acid position 457 (e.g., G457G⁶, G457fs*6³³)
Amino acid position 469 (e.g., C469G)⁴¹
Amino acid position 478 (e.g., H478H)⁴¹
Amino acid position 500 (e.g., Y500H)⁶
Amino acid position 525 (e.g., R525X)⁴
Δ Amino acid position 529⁶
Amino acid position 535 (e.g., H535L⁶, H535N⁴¹)
Amino acid position 553 (e.g., P553P)⁴³
Amino acid position 554 (e.g., D554N^{1,6}, D554A³⁵)
Δ Amino acid positions 556-628⁴⁴
Δ Amino acid positions 559-563³⁵
Amino acid position 570 (e.g., L570L)⁴¹
Amino acid position 577 (e.g., I577V)¹⁹
Amino acid position 581 (e.g., E581K)³⁵
Amino acid positions 554 and 581 (e.g., D554A + E581K)³⁵
Amino acid position 585 (e.g., E585X)²¹
Amino acid position 600 (e.g., R600W^{2,4}, R600Q⁶)
Amino acid position 602 (e.g., R602X)^{3,6}
Amino acid position 628 (e.g., R628W)⁶
Amino acid position 631 (e.g., R631Q)²⁸
Δ Amino acid positions 645-699⁴
Amino acid position 661 (e.g., I661T)^{1,4,6}
Amino acid position 665 (e.g., E665X)^{4,6}
Amino acid position 672 (e.g., K672fs⁶, K672Vfs*1³⁵)
Amino acid position 674 (e.g., M674T)¹⁹
Amino acid positions 78 and 674 (e.g., H78Q/M674T)¹⁹
Amino acid position 684 (e.g., D684D)⁴¹
Amino acid position 688 (e.g., D688G)⁶
Amino acid position 694 (e.g., I694T⁶, I694N¹⁷)
Amino acid position 695 (e.g., E695K)²⁷
Amino acid position 709 (e.g., K709fs⁶, K709Qfs*41¹³)
Amino acid position 717 (e.g., T717N)⁴
Amino acid position 733 (e.g., G733R)⁶
Amino acid position 757 (e.g., Y757X)⁴
Amino acid position 749 (e.g., L749P)²¹
Amino acid position 792 (e.g., P792fs)⁶
Δ Amino acid position 795-797⁶
Amino acid position 809 (e.g., I809L)²⁷
Amino acid position 814 (e.g., K814N)²⁸
Amino acid position 833 (e.g., R833Q²⁷, R833W⁴¹)
Amino acid position 835 (e.g., K835Rfs*36)³⁵
Amino acid position 845 (e.g., K845fs)²⁵
Amino acid position 849 (e.g., R849Q)²⁴
Amino acid position 853 (e.g., F853S, F853fs)⁶
Amino acid position 867 (e.g., R867C¹, R867fs⁶, R867H²³)
Amino acid position 885 (e.g., K885T)⁴¹
Amino acid position 888 (e.g., T888T)⁴¹
Amino acid position 892 (e.g., G892R)⁶
Amino acid position 912 (e.g., G912R)³⁵
Amino acid position 921 (e.g., S921S)⁴¹
Amino acid position 924 (e.g., Y924C)²⁸
Amino acid position 930 (e.g., R930X⁶, R930Q²⁸)
Amino acid position 941 (e.g., R941X)³⁵
Amino acid position 946 (e.g., R946T)⁴¹
Amino acid position 952 (e.g., R952Q^{5,9,15}, R952X⁶)
Amino acid position 958 (e.g., N958fs)⁶
Amino acid position 960 (e.g., A960A)⁴¹
Δ Amino acid position 971⁴³
Amino acid position 976 (e.g., A976E⁴¹, A976A⁴³)
Amino acid position 981 (e.g., E981K)²⁰
Amino acid position 994 (e.g., S994R)⁴
Amino acid position 1011 (e.g., L1011fs*18)³³
Amino acid position 1012 (e.g., S1012I)¹⁰
Amino acid position 1014 (e.g., R1014X)^{6,11}
Amino acid position 1015 (e.g., F1015L)²⁷
Amino acid position 1023 (e.g., Q1023fs)⁶
Amino acid position 1040 (e.g., G1040R)^{1,6}
Amino acid position 1044 (e.g., S0144L)³⁴
Amino acid position 1047 (e.g., L1047fs)⁶
Amino acid position 1050 (e.g., I1050K)³¹
Amino acid position 1052 (e.g., L1052R)²⁸
Amino acid position 1095 (e.g., W1095X)¹¹
Amino acid position 1098 (e.g., V1098X)³⁵
Amino acid position 1131 (e.g., Q1131X)⁴⁴
Amino acid position 1142 (e.g., A1142Tfs*35)⁴³
Amino acid position 1144 (e.g., Y1144Y)⁴³
Amino acid position 1150 (e.g., I1150T)⁴¹
Amino acid position 1152 (e.g., A1152T)³⁰
Amino acid position 1159 (e.g., P1159P)^{25,43}
Amino acid position 1164 (e.g., R1164X)⁶
Amino acid position 1193 (e.g., R1193fs*39)³³
Amino acid position 1197 (e.g., V1197L)⁴¹
Amino acid position 1208 (e.g., A1208fs)⁶
Amino acid position 1209 (e.g., Y1209Lfs*28)⁴
Amino acid position 1211 (e.g., F1211L)²⁷
Amino acid position 1219 (e.g., D1219H⁵, D1219G²⁷)
Amino acid position 1223 (e.g., S1223S)⁴¹
Amino acid position 1233 (e.g., P1233P)⁴¹
Amino acid position 1241 (e.g., G1241fs)⁶
Amino acid position 1248 (e.g., T1248T)⁴³
Splice site mutation IVS3 + 1 + 3delGTG⁶
Splice site mutation IVS3 − 2A > G⁶
IVS6 + 5T > G^{17,25}
Splice site mutation IVS8 + 1G > T⁶
IVS9 − G > A²⁶
IVS12 + 1G > A²⁵
Splice site mutation IVS17 − 1G > A⁶
Splice site mutation IVS18 + 2T > C⁶
Splice site mutation IVS20 − 4CT > AA
Splice site mutation IVS21 + 5G > A⁶
Splice site mutation IVS23 − 3C > A⁶
Splice site mutation IVS26 + 2T > A⁶
g.24774-42062del⁴
c.-4C > G⁴¹
c.145C > T¹²
c.181 − 72G > A⁹
c.182 − 5T > A⁴¹
c.182 − 72G > A⁴¹
c.246A > G⁹
c.239G > A³⁹
c.279 + 1_279 + 3delGTG⁴⁶
c.280 − 2A > G⁴⁶
c.625_62715delinsACAGTAAT⁴⁶
c.554 + 122C > T⁹
c.555 − 3T > C²⁷
c.625 + 5 G > T⁴
Amino acid position 209 (e.g., P209T) and c.625 + 5 G > T⁴
c.628 − 30G > A⁴¹
c.628 − 31C > T⁴¹
c.698 + 1G > T⁴⁶
c.698 + 20C > T⁴¹
c.782 − 1G > A⁴⁶
c.782 − 34G > A⁴¹
Δ795-797¹⁴
c.782 − 1G > A⁴
c.852A > C²⁷
c.941 − 1G > A⁴⁶
c.1014C > T⁹
c.1029 + 35G > A⁹
c.1221-8C.G⁴¹
1226delA¹⁶
c.1429 + 1G > A⁴⁶
c.1429 + 2T > G¹³
c.1429 + 49G > A⁴¹
c.1430 − 42A > G⁴¹
c.1493T > C¹²
c.1587_1589delCTT⁴⁶
c.1630 + 2T > G²⁷
c.1631 − 10T > A⁴¹
c.1637 − 37T > C⁴¹
1660 G > A¹⁴
1798 C > T¹⁴
1799 G > A¹⁴
c.1819 − 39_41delAA⁹
c.1819 + 1G > A³¹
c.1820 − 27G > A⁴¹
c.1918 + 8C > T²⁷
c.1933 − 1G > AK46
c.2097 + 2T > C³²
c.2097 + 60T > G⁴¹
c.2097 + 89T > C⁴¹
c.2097 + 97T > G⁴¹
c.2210 − 114T > C⁹
2210delA¹⁶
c.2210 − 45_50dupATAAAA⁹
c.2285 + 29C · T⁴¹
c.2285 + 32A > G⁴¹
c.2286 − 4_2286-3delinsAA⁴⁶
c.2418 + 5G > A⁴⁶
c.2707 + 3G > C²⁷
c.2707 + 9T > G⁴¹
c.2707 + 43A > G⁴¹
c.2709 − 59T > C⁴¹
c.2931 + 9A > G⁴¹
c.2931 + 59T > A⁴¹
C.2932 − 3C > A⁴⁶
c.2932 + 59T > A⁹
c.2937A > C²⁷
c.3016 − 9C > A³¹
c.3033-3034del¹⁹
3122delTCCTA/insACATCGATGTTGATGTTAGG⁴⁵
3318 G > A¹⁴
c.3400 + 2T > A⁴⁶
c.3401 − 175C > T⁹
c.3401 − 167C > T⁹
c.3401 − 108C > T⁹
c.3531 + 8G > T^{9,15}
c.3532 − 15C > T⁹
Δ Phe ex 15⁴
Ex1_Ex13del⁶
Ex2_Ex6del³³
Ex12_Ex14del²⁷
Skipped Exon 24⁴⁵
del5′UTR-ex18¹¹
c.*11C > T⁴¹
c.*1101 + 366G > A⁷
g.92918del565³¹
GC preceding exon 16 (e.g., resulting in a 4 bp deletion)⁴²
Frameshift from the 5′ end of exon 16⁴²
5′ 1.4 kb deletion⁴⁶

TABLE 2
Selected ATP8B1 Mutations Associated with PFIC-1

Amino acid position 23 (e.g., P23L)⁵
Amino acid position 78 (e.g., H78Q)¹⁹
Amino acid position 93 (e.g., A93A)⁶
Amino acid position 96 (e.g., A96G)²⁷
Amino acid position 127 (e.g., L127P)⁶
Amino acid position 197 (e.g., G197Lfs*10)²²
Amino acid position 205 (e.g., N205fs)⁶
Amino acid position 209 (e.g., P209T)⁴
Amino acid position 233 (e.g., G233R)³⁸
Amino acid position 243 (e.g., L243fs*28)³³
Amino acid position 288 (e.g., L288S)⁶
Amino acid position 296 (e.g., R296C)¹¹
Amino acid position 308 (e.g., G308V^{1,6})
Amino acid position 320 (e.g., M320Vfs*13)¹¹
Amino acid position 403 (e.g., S403Y)⁶
Amino acid position 407 (e.g., S407N)⁴⁰
Amino acid position 412 (e.g., R412P)⁶
Amino acid position 415 (e.g., Q415R)²⁷
Amino acid position 429 (e.g., E429A)⁶
Amino acid position 446 (e.g., G446R)⁴
Amino acid position 456 (e.g., T456M)^{3,6}
Amino acid position 457 (e.g., G457G⁶, G457fs*6³³)
Amino acid position 500 (e.g., Y500H)⁶
Amino acid position 525 (e.g., R525X)⁴
Δ Amino acid position 529⁶
Amino acid position 535 (e.g., H535L)⁶
Amino acid position 554 (e.g., D554N)^{1,6}
Amino acid position 577 (e.g., I577V)¹⁹
Amino acid position 585 (e.g., E585X)²¹
Amino acid position 600 (e.g., R600W)⁴
Amino acid position 602 (e.g., R602X)^{3,6}
Amino acid position 661 (e.g., I661T)^{4,6}
Amino acid position 665 (e.g., E665X)^{4,6}
Δ Amino acid positions 645-699⁴
Amino acid position 672 (e.g., K672fs)⁶
Amino acid position 674 (e.g., M674T)¹⁹
Amino acid positions 78 and 674 (e.g., H78Q/M674T)¹⁹
Amino acid position 688 (e.g., D688G)⁶
Amino acid position 694 (e.g., I694N)¹⁷
Amino acid position 695 (e.g., E695K)²⁷
Amino acid position 709 (e.g., K709fs)⁶
Amino acid position 717 (e.g., T717N)⁴
Amino acid position 733 (e.g., G733R)⁶
Amino acid position 749 (e.g., L749P)²¹
Amino acid position 757 (e.g., Y757X)⁴
Amino acid position 792 (e.g., P792fs)⁶
Amino acid position 809 (e.g., I809L)²⁷
Amino acid position 853 (e.g., F853S, F853fs)⁶
Amino acid position 867 (e.g., R867fs)⁶
Amino acid position 892 (e.g., G892R)⁶
Amino acid position 930 (e.g., R930X⁶, R952Q¹⁵)
Amino acid position 952 (e.g., R952X)⁶
Amino acid position 958 (e.g., N958fs)⁶
Amino acid position 981 (e.g., E981K)²⁰
Amino acid position 994 (e.g., S994R)⁴
Amino acid position 1014 (e.g., R1014X)^{6,11}
Amino acid position 1015 (e.g., F1015L)²⁷
Amino acid position 1023 (e.g., Q1023fs)⁶
Amino acid position 1040 (e.g., G1040R)^{1,6}
Amino acid position 1047 (e.g., L1047fs)⁶
Amino acid position 1095 (e.g., W1095X)¹¹
Amino acid position 1208 (e.g., A1208fs)⁶
Amino acid position 1209 (e.g., Y1209Lfs*28)⁴
Amino acid position 1211 (e.g., F1211L)²⁷
Amino acid position 1219 (e.g., D1219H⁵, D1219G²⁷)
Splice site mutation IVS3 + 1_+3delGTG⁶
Splice site mutation IVS3 − 2A > G⁶
IVS6 + 5T > G¹⁷
Splice site mutation IVS8 + 1G > T⁶
IVS9 − G > A²⁶
Splice site mutation IVS17 − 1G > A⁶
Splice site mutation IVS18 + 2T > C⁶
Splice site mutation IVS21 + 5G > A⁶
g.24774-42062del⁴
c.145C > T¹²
c.239G > A³⁹
c.625 + 5 G > T⁴
Amino acid position 209 (e.g., P209T) and c.625 + 5 G > T⁴
c.782 − 1G > A⁴
c.1493T > C¹²
c.1630 + 2T > G²⁷
1660 G > A¹⁴
c.2707 + 3G > C²⁷
c.2097 + 2T > C³²
c.3033-3034del¹⁹
3318 G > A¹⁴
c.3158 + 8G > T¹⁵
Δ Phe ex 15⁴
Ex1_Ex13del⁶
Ex2_Ex6del³³
Ex12_Ex14del²⁷
del5′UTR-ex18¹¹
c.*1101 + 366G > A⁷
GC preceding exon 16 (e.g., resulting in a 4 bp deletion)⁴²
Frameshift from the 5′ end of exon 16⁴²

^{A} A mutation to ‘X’ denotes an early stop codon

### References for Tables 1 and 2

¹ Folmer et al., Hepatology. 2009, vol. 50(5), p. 1597-1605.
² Hsu et al., Hepatol Res. 2009, vol. 39(6), p. 625-631.
³ Alvarez et al., Hum Mol Genet. 2004, vol. 13(20), p. 2451-2460.
⁴ Davit-Spraul et al., Hepatology 2010, vol. 51(5), p. 1645-1655.
⁵ Vitale et al., J Gastroenterol. 2018, vol. 53(8), p. 945-958.
⁶ Klomp et al., Hepatology 2004, vol. 40(1), p. 27-38.
⁷ Zarenezhad et al., Hepatitis Monthly: 2017, vol. 17(2); e43500.
⁸ Dixon et al., Scientific Reports 2017, vol. 7, 11823.
⁹ Painter et al., Eur J Hum Genet. 2005, vol. 13(4), p. 435-439.
¹⁰ Deng et al., World J Gastroenterol. 2012, vol. 18(44), p. 6504-6509.
¹¹ Giovannoni et al., PLoS One. 2015, vol. 10(12): e0145021.
¹² Li et al., Hepatology International 2017, vol. 11, No. 1, Supp. Supplement 1, pp. S180. Abstract Number: OP284.
¹³ Togawa et al., Journal of Pediatric Gastroenterology and Nutrition 2018, vol. 67, Supp. Supplement 1, pp. S363. Abstract Number: 615.
¹⁴ Miloh et al., Gastroenterology 2006, vol. 130, No. 4, Suppl. 2, pp. A759-A760. Meeting Info.: Digestive Disease Week Meeting/107th Annual Meeting of the American-Gastroenterological-Association. Los Angeles, CA, USA. May 19.
¹⁵ Dröge et al., Zeitschrift fur Gastroenterologie 2015, vol. 53, No. 12. Abstract Number: A3-27. Meeting Info: 32. Jahrestagung der Deutschen Arbeitsgemeinschaft zum Studium der Leber. Dusseldorf, Germany. 22-1-2016-23-1-2016
¹⁶ Mizuochi et al., Clin Chim Acta. 2012, vol. 413(15-16), p. 1301-1304.
¹⁷ Liu et al., Hepatology International 2009, vol. 3, No. 1, p. 184-185. Abstract Number: PE405. Meeting Info: 19th Conference of the Asian Pacific Association for the Study of the Liver. Hong Kong, China. 13-2-2009-16-2-2009
¹⁸ McKay et al., Version 2. F1000Res. 2013; 2: 32. DOI: 10.12688/f1000research.2-32.v2
¹⁹ Hasegawa et al., Orphanet J Rare Dis. 2014, vol. 9:89.
²⁰ Stone et al., J Biol Chem. 2012, vol. 287(49), p. 41139-51.
²¹ Kang et al., J Pathol Transl Med. 2019-5-16. doi: 10.4132/jptm.2019.05.03. [Epub ahead of print]
²² Sharma et al., BMC Gastroenterol. 2018, vol. 18(1), p. 107.
²³ Uegaki et al., Intern Med. 2008, vol. 47(7), p. 599-602.
²⁴ Goldschmidt et al., Hepatol Res. 2016, vol. 46(4), p. 306-311.
²⁵ Liu et al., J Pediatr Gastroenterol Nutr. 2010, vol. 50(2), p. 179-183.
²⁶ Jung et al., J Pediatr Gastroenterol Nutr. 2007, vol. 44(4), p. 453-458.
²⁷ Bounford. University of Birmingham. Dissertation Abstracts International, (2016) Vol. 75, No. 1C.

Order No.: AA110588329. ProQuest Dissertations & Theses.
²⁸ Stolz et al., Aliment Pharmacol Ther. 2019, vol. 49(9), p. 1195-1204.
²⁹ Ivashkin et al., Hepatology International 2016, vol. 10, No. 1, Supp. SUPPL. 1, pp. S461. Abstract Number: LBO-38. Meeting Info: 25th Annual Conference of the Asian Pacific Association for the Study of the Liver, APASL 2016. Tokyo, Japan. 20-2-2016-24-2-2016
³⁰ Blackmore et al., J Clin Exp Hepatol. 2013, vol. 3(2), p. 159-161.
³¹ Matte et al., J Pediatr Gastroenterol Nutr. 2010, vol. 51(4), p. 488-493.
³² Squires et al., J Pediatr Gastroenterol Nutr. 2017, vol. 64(3), p. 425-430.
³³ Hayshi et al., EBioMedicine. 2018, vol. 27, p. 187-199.
³⁴ Nagasaka et al., J Pediatr Gastroenterol Nutr. 2007, vol. 45(1), p. 96-105.
³⁵ Wang et al., PLoS One. 2016; vol. 11(4): e0153114.
³⁶ Narchi et al., Saudi J Gastroenterol. 2017, vol. 23(5), p. 303-305.
³⁷ Alashkar et al., Blood 2015, vol. 126, No. 23. Meeting Info.: 57th Annual Meeting of the American-Society-of-Hematology. Orlando, FL, USA. Dec. 5-8, 2015. Amer Soc Hematol.
³⁸ Ferreira et al., Pediatric Transplantation 2013, vol. 17, Supp. SUPPL. 1, pp. 99. Abstract Number: 239. Meeting Info: IPTA 7th Congress on Pediatric Transplantation. Warsaw, Poland. 13-7-2013-16-7-2013.
³⁹ Pauli-Magnus et al., J Hepatol. 2005, vol. 43(2), p. 342-357.
⁴⁰ Jericho et al., Journal of Pediatric Gastroenterology and Nutrition 2015, vol. 60(3), p. 368-374.
⁴¹ van der Woerd et al., PLoS One. 2013, vol. 8(11): e80553.
⁴² Copeland et al., J Gastroenterol Hepatol. 2013, vol. 28(3), p. 560-564.
⁴³ Droge et al., J Hepatol. 2017, vol. 67(6), p. 1253-1264.
⁴⁴ Chen et al., Journal of Pediatrics 2002, vol. 140(1), p. 119-124.
⁴⁵ Jirsa et al., Hepatol Res. 2004, vol. 30(1), p. 1-3.
⁴⁶ van der Woerd et al., Hepatology 2015, vol. 61(4), p. 1382-1391.

In some embodiments, the mutation in ATP8B1 is selected from L127P, G308V, T456M, D554N, F529del, 1661T, E665X, R930X, R952X, R1014X, and G1040R.

TABLE 3
Exemplary ABCB11 Mutations

Amino acid position 1 (e.g., M1V)⁹
Amino acid position 4 (e.g., S4X)^{A,64}
Amino acid position 8 (e.g., R8X)⁸⁸
Amino acid position 19 (e.g., G19R)⁵⁶
Amino acid position 24 (e.g., K24X)³⁵
Amino acid position 25 (e.g., S25X)^{5,14}
Amino acid position 26 (e.g., Y26Ifs*7)³⁸
Amino acid position 36 (e.g., D36D)²⁷
Amino acid position 38 (e.g., K38Rfs*24)⁷³
Amino acid position 43 (e.g., V43I)⁵⁷
Amino acid position 49 (e.g., Q49X)⁷³
Amino acid position 50 (e.g., L50S, L50W)⁵⁷
Amino acid position 52 (e.g., R52W²⁶, R52R²⁸)
Amino acid position 56 (e.g., S56L)⁵⁸
Amino acid position 58 (e.g., D58N)⁶²
Amino acid position 62 (e.g., M62K)⁹
Amino acid position 66 (e.g., S66N)¹⁷
Amino acid position 68 (e.g., C68Y)⁴¹
Amino acid position 50 (e.g., L50S)^{5,7}
Amino acid position 71 (e.g., L71H)⁷³
Amino acid position 74 (e.g., I74R)⁷¹
Amino acid position 77 (e.g., P77A)⁷³
Amino acid position 87 (e.g., T87R)⁶⁷
Amino acid position 90 (e.g., F90F)^{7,27}
Amino acid position 93 (e.g., Y93S¹³, Y93X⁸⁸)
Amino acid position 96 (e.g., E96X)⁸⁸
Amino acid position 97 (e.g., L97X)³⁹
Amino acid position 101 (e.g., Q101Dfs*8)⁹
Amino acid position 107 (e.g., C107R)³⁶
Amino acid position 112 (e.g., I112T)⁹
Amino acid position 114 (e.g., W114R)^{2,9}
Amino acid position 123 (e.g. M123T)⁶⁷
Amino acid position 127 (e.g., T127Hfs*6)⁵
Amino acid position 129 (e.g., C129Y)²⁵
Amino acid position 130 (e.g., G130G)⁷⁷
Amino acid position 134 (e.g., I134I)²⁸
Amino acid position 135 (e.g., E135K^{7,13}, E135L¹⁷)
Amino acid position 137 (e.g., E137K)⁷
Amino acid position 157 (e.g., Y157C)⁵
Amino acid position 161 (e.g., C161X)³⁹
Amino acid position 164 (e.g., V164Gfs*7³⁰, V164I⁸⁵)
Amino acid position 167 (e.g., A167S⁴, A167V⁷, A167T^{9,17})
Amino acid position 181 (e.g., R181I)³⁵
Amino acid position 182 (e.g., I182K)⁹
Amino acid position 183 (e.g., M183V⁸, M183T⁹)
Amino acid position 185 (e.g., M185I)⁷³
Amino acid position 186 (e.g., E186G)^{2,7,22}
Amino acid position 188 (e.g., G188W)⁷³
Amino acid position 194 (e.g., S194P)⁷
Amino acid position 198 (e.g., L198P)⁷
Amino acid position 199 (e.g., N199Ifs*15X)⁸⁸
Amino acid position 206 (e.g., 1206V)²⁸
Amino acid position 212 (e.g., A212T)⁷³
Amino acid position 217 (e.g., M217R)⁸⁸
Amino acid position 225 (e.g., T225P)⁵⁷
Amino acid position 226 (e.g., S226L)⁹
Amino acid position 232 (e.g., L232Cfs*9)⁹
Amino acid position 233 (e.g., L233S)⁸⁶
Amino acid position 238 (e.g., G238V)^{2,7}
Amino acid position 242 (e.g., T242I)^{5,7}
Amino acid position 245 (e.g., I245Tfs*26)⁵⁷
Amino acid position 256 (e.g., A256G)⁹
Amino acid position 260 (e.g., G260D)⁷
Amino acid position 269 (e.g., Y269Y)²⁷
Amino acid position 277 (e.g., A277E)⁷⁷
Amino acid position 283 (e.g., E283D)⁷³
Amino acid positions 212 and 283 (e.g., A212T + E283D)⁷³
Amino acid position 284 (e.g., V284L^{7,39}, V284A⁷, V284D²³)
Amino acid position 297 (e.g., E297G^{1,2,5,7}, E297K⁷)
Amino acid position 299 (e.g., R299K)²⁸
Amino acid position 303 (e.g., R303K⁸, R303M⁶³ R303fsX321⁸³)
Amino acid position 304 (e.g., Y304X)²⁶
Amino acid position 312 (e.g., Q312H)⁷
Amino acid position 313 (e.g., R313S)^{5,7}
Amino acid position 314 (e.g., W314X)⁵⁷
Amino acid position 318 (e.g., K318Rfs*26)²⁹
Amino acid position 319 (e.g., G319G)⁷
Amino acid position 327 (e.g., G327E)^{5,7}
Amino acid position 330 (e.g., W330X)²⁴
Amino acid position 336 (e.g., C336S)^{2,7}
Amino acid position 337 (e.g., Y337H)^{21,27}
Amino acid position 342 (e.g., W342G)⁵⁰
Amino acid position 354 (e.g., R354X)⁹
Amino acid position 361 (e.g., Q361X⁵⁷, Q361R⁷⁴)
Amino acid position 366 (e.g., V366V²⁸, V366D⁵⁷)
Amino acid position 368 (e.g., V368Rfs*27)⁵
Amino acid position 374 (e.g., G374S)³
Amino acid position 380 (e.g., L380Wfs*18)⁵
Amino acid position 382 (e.g., A382G)⁸⁸
Δ Amino acid positions 382-388⁵
Δ Amino acid positions 383-389⁵⁷
Amino acid position 387 (e.g., R387H)⁹
Amino acid position 390 (e.g., A390P)^{5,7}
Amino acid position 395 (e.g., E395E)²⁸
Amino acid position 404 (e.g., D404G)⁹
Amino acid position 410 (e.g., G410D)^{5,7}
Amino acid position 413 (e.g., L413W)^{5,7}
Amino acid position 415 (e.g., R415X)⁴²
Amino acid position 416 (e.g., I416I)²⁷
Amino acid position 420 (e.g., I420T)⁹
Amino acid position 423 (e.g., H423R)¹³
Amino acid position 432 (e.g., R432T)^{1,2,7}
Amino acid position 436 (e.g., K436N)⁴⁰
Amino acid position 440 (e.g., D440E)⁸⁸
Amino acid position 444 (e.g., V444A)²
Amino acid position 454 (e.g., V454X)⁴⁹
Amino acid position 455 (e.g., G455E)⁹
Amino acid position 457 (e.g., S457Vfs*23)⁸⁸
Amino acid position 461 (e.g., K461E)^{2,7}
Amino acid position 462 (e.g., S462R)⁸⁸
Amino acid position 463 (e.g., T463I)^{5,7}
Amino acid position 466 (e.g., Q466K)^{5,7}
Amino acid position 470 (e.g., R470Q^{5,7}, R470X⁹)
Amino acid position 471 (e.g., Y472X)⁵
Amino acid position 472 (e.g., Y472C^{5,27}, Y472X¹⁴)
Amino acid position 473 (e.g., D473Q³⁵, D473V⁸⁸)
Amino acid position 475 (e.g., C475X)²⁹
Amino acid position 481 (e.g., V481E)^{5,7}
Amino acid position 482 (e.g., D482G)^{2,5,7}
Amino acid position 484 (e.g., H484Rfs*5)⁹
Amino acid position 487 (e.g., R487H², R487P⁵)
Amino acid position 490 (e.g., N490D)^{5,7}
Amino acid position 493 (e.g., W493X)⁸
Amino acid positon 496 (e.g., D496V)⁸⁸
Amino acid position 498 (e.g., I498T)^{2,7}
Amino acid position 499 (e.g., G499E)⁷³
Amino acid position 501 (e.g., V501G)⁶⁸
Amino acid position 504 (e.g., E504K)⁷⁹
Amino acid position 510 (e.g., T510T)⁷
Amino acid position 512 (e.g., I512T)^{5,7}
Amino acid position 515 (e.g., N515T^{5,7}, N515D⁶⁴)
Amino acid position 516 (e.g., I516M)¹⁷
Amino acid position 517 (e.g., R517H)^{5,7}
Amino acid position 520 (e.g., R520X)⁵
Amino acid position 523 (e.g., A523G)¹³
Amino acid position 528 (e.g., I528Sfs*21⁵, I528X⁹, I528T⁷³)
Amino acid position 535 (e.g., A535A⁷, A535X⁸⁹)
Amino acid position 540 (e.g., F540L)⁴⁶
Amino acid position 541 (e.g., I541L^{5,7}, I541T^{5,17})
Amino acid position 546 (e.g., Q546K³⁹, Q546H⁷³)
Amino acid position 548 (e.g., F548Y)^{5,7}
Amino acid position 549 (e.g., D549V)⁹
Amino acid position 554 (e.g., E554K)²¹
Amino acid position 556 (e.g., G556R)⁶⁷
Amino acid position 558 (e.g., Q558H)²³
Amino acid position 559 (e.g., M559T)⁵⁷
Amino acid position 562 (e.g., G562D^{5,7}, G562S⁷³)
Amino acid position 570 (e.g., A570T^{2,5,7}, A570V²⁶)
Amino acid position 575 (e.g., R575X^{2,5}, R575Q²¹)
Amino acid position 580 (e.g., L580P)⁵⁷
Amino acid position 586 (e.g., T586I)⁷
Amino acid position 587 (e.g., S587X)⁷³
Amino acid position 588 (e.g., A588V^{5,7}, A588P⁷³)
Amino acid position 591 (e.g., N591S)^{2,7}
Amino acid position 593 (e.g., S593R)^{2,7}
Amino acid position 597 (e.g., V597V⁹, V597L¹³)
Amino acid position 603 (e.g., K603K)⁵⁵
Amino acid position 609 (e.g., H609Hfs*46)²⁶
Amino acid position 610 (e.g., I610Gfs*45⁹, I610T⁵⁷)⁹
Amino acid position 615 (e.g., H615R)²⁶
Amino acid position 616 (e.g., R616G²⁸, R616H⁷³)
Amino acid position 619 (e.g., T619A)²⁸
Amino acid position 623 (e.g., A623A)²⁸
Amino acid position 625 (e.g., T625Nfs*5)²⁶
Amino acid position 627 (e.g., I627T)⁷
Amino acid position 628 (e.g., G628Wfs*3)⁷⁰
Amino acid position 636 (e.g., E636G)²
Amino acid position 648 (e.g., G648Vfs*6⁵, G648V⁵⁰)
Amino acid position 655 (e.g., T655I)⁷
Amino acid position 669 (e.g., I669V)²⁶
Amino acid position 676 (e.g., D676Y)¹¹
Amino acid position 677 (e.g., M677V)^{7,13}
Amino acid position 679 (e.g., A679V)⁵⁸
Amino acid position 685 (e.g., G685W)⁶⁰
Amino acid position 696 (e.g., R696W²⁷, R696Q⁵⁸)
Amino acid position 698 (e.g., R698H^{7,9}, R698K⁶¹, R698C⁸⁸)
Amino acid position 699 (e.g., S699P)⁹
Amino acid position 701 (e.g., S701P)⁵⁸
Amino acid position 702 (e.g., Q702X)⁸⁹
Amino acid position 709 (e.g., E709K)⁷
Amino acid position 710 (e.g., P710P)⁷
Amino acid position 712 (e.g., L712L)²⁸
Amino acid position 721 (e.g., Y721C)⁸⁸
Amino acid position 729 (e.g., D724N)³⁹
Amino acid position 731 (e.g., P731S)²³
Amino acid position 740 (e.g., P740Qfs*6)⁷³
Amino acid position 758 (e.g., G758R)⁵
Amino acid position 766 (e.g., G766R)^{5,24}
Amino acid position 772 (e.g., Y772X)⁵
Amino acid position 804 (e.g., A804A)⁷
Amino acid position 806 (e.g., G806D⁴⁴, G806G⁵⁵)
Amino acid position 809 (e.g., S809F)⁸¹
Amino acid position 817 (e.g., G817G)⁸⁸
Amino acid position 818 (e.g., Y818F)⁷
Amino acid position 824 (e.g., G824E)⁴²
Amino acid position 825 (e.g., G825G)⁷³
Amino acid position 830 (e.g., R830Gfs*28)⁷³
Amino acid position 832 (e.g., R832C^{7,26}, R832H⁴¹)
Amino acid position 842 (e.g., D842G)²
Amino acid position 848 (e.g., D848N)⁷³
Amino acid position 855 (e.g., G855R)¹¹
Amino acid position 859 (e.g., T859R)^{5,7}
Amino acid position 865 (e.g., A865V)²⁷
Amino acid position 866 (e.g., S866A)⁵⁷
Amino acid position 868 (e.g., V868D)⁷³
Amino acid position 869 (e.g., Q869P)⁷³
Amino acid position 875 (e.g., Q875X)⁷³
Amino acid position 877 (e.g., G877R)⁵⁶
Amino acid position 879 (e.g., I879R)⁸⁸
Amino acid position 893 (e.g., A893V)⁵⁷
Amino acid position 901 (e.g., S901R¹⁷, S901I⁷³)
Amino acid position 903 (e.g., V903G)⁵⁷
Δ Amino acid position 919¹²
Amino acid position 923 (e.g., T923P)^{2,7}
Amino acid position 926 (e.g., A926P)^{2,7}
Amino acid position 928 (e.g., R928X¹⁵, R928Q⁴⁰)
Amino acid position 930 (e.g., K930X⁵, K930Efs*79^{5,10}, K930Efs*49²⁶)
Amino acid position 931 (e.g., Q931P)²⁷
Amino acid position 945 (e.g., S945N)⁵⁷
Amino acid position 948 (e.g., R948C)^{5,7,26}
Amino acid position 958 (e.g., R958Q)²⁸
Amino acid position 969 (e.g., K969K)⁸⁸
Δ Amino acid positions 969-972⁵
Amino acid position 973 (e.g., T973I)⁵⁷
Amino acid position 976 (e.g., Q976R⁵⁸, Q976X⁸⁸)
Amino acid position 979 (e.g., N979D)^{5,7}
Amino acid position 981 (e.g., Y981Y)²⁸
Amino acid position 982 (e.g., G982R)^{2,5,7}
Amino acid positions 444 and 982 (e.g., V444A + G982R)³⁸
Amino acid position 995 (e.g., A995A)²⁸
Amino acid position 1001 (e.g., R1001R)⁹
Amino acid position 1003 (e.g., G1003R)²⁴
Amino acid position 1004 (e.g., G1004D)^{2,7}
Amino acid position 1027 (e.g., S1027R)²⁶
Amino acid position 1028 (e.g., A1028A^{7,10,88}, A1028E⁸⁸)
Amino acid position 1029 (e.g., T1029K)⁵
Amino acid position 1032 (e.g., G1032R)¹²
Amino acid position 1041 (e.g., Y1041X)⁹
Amino acid position 1044 (e.g., A1044P)⁸⁸
Amino acid position 1050 (e.g., R1050C)^{2,7,57}
Amino acid position 1053 (e.g., Q1053X)⁵⁷
Amino acid position 1055 (e.g., L1055P)³⁶
Amino acid position 1057 (e.g., R1057X², R1057Q⁵⁸)
Amino acid position 1058 (e.g., Q1058Hfs*38⁹, Q1058fs*38¹⁷,
Q1058X⁷³)
Amino acid position 1061 (e.g., I1061Vfs*34)⁹
Amino acid position 1083 (e.g., C1083Y)⁴⁷
Amino acid position 1086 (e.g., T1086T)²⁸
Amino acid position 1090 (e.g., R1090X)^{2,5}
Amino acid position 1099 (e.g., L1099Lfs*38)²⁶
Amino acid position 1100 (e.g., S1100Qfs*38)¹³
Amino acid position 1110 (e.g., A1110E)^{5,7}
Amino acid position 1112 (e.g., V1112F)⁷⁰
Amino acid position 1116 (e.g., G1116R⁷, G1116F^{9,17}, G1116E³⁶)
Amino acid position 1120 (e.g., S1120N)⁸⁸
Amino acid position 1128 (e.g., R1128H^{2,7}, R1128C^{5,7,13})
Amino acid position 1131 (e.g., D1131V)²⁷
Amino acid position 1144 (e.g., S1144R)⁷
Amino acid position 1147 (e.g., V1147X)⁵
Amino acid position 1153 (e.g., R1153C^{2,5,7}, R1153H⁵)
Amino acid position 1154 (e.g., S1154P)^{5,7}
Amino acid position 1162 (e.g., E1162X)³⁹
Δ Amino acid position 1165⁸⁸
Amino acid position 1164 (e.g., V1164Gfs*7)
Amino acid position 1173 (e.g., N1173D)⁵⁷
Amino acid position 1175 (e.g., K1175T)⁵⁸
Amino acid position 1186 (e.g., E1186K)⁷
Amino acid position 1192 (e.g., A1192Efs*50)⁹
Amino acid position 1196 (e.g., Q1196X)⁸⁸
Amino acid position 1197 (e.g., L1197G)⁷
Amino acid position 1198 (e.g., H1198R)²⁷
Amino acid position 1204 (e.g., L1204P)⁸⁸
Amino acid position 1208 (e.g. Y1208C)⁷³
Amino acid position 1210 (e.g., T1210P^{5,7}, T1210F⁵⁷)
Amino acid position 1211 (e.g., N1211D)⁷
Amino acid position 1212 (e.g., V1212F)³⁶
Amino acid position 1215 (e.g., Q1215X)⁵
Amino acid position 1221 (e.g., R1221K)⁵³
Amino acid position 1223 (e.g., E1223D)⁷
Amino acid position 1226 (e.g., R1226P)⁷³
Amino acid position 1228 (e.g., A1228V)⁷
Amino acid position 1231 (e.g., R1231W^{5,7}, R1231Q^{5,7})
Amino acid position 1232 (e.g., A1232D)¹⁷
Amino acid position 1235 (e.g., R1235X)^{5,12}
Amino acid position 1242 (e.g., L1242I)^{5,7}
Amino acid position 1243 (e.g., D1243G)⁶⁷
Amino acid position 1249 (e.g., L1249X)⁷³
Amino acid position 1256 (e.g., T1256fs*1296)⁸³
Amino acid position 1268 (e.g., R1268Q)^{2,7}
Amino acid position 1276 (e.g., R1276H)³⁰
Amino acid position 1283 (e.g., A1283A²⁸, A1283V⁸⁸)
Amino acid position 1292 (e.g., G1292V)⁷³
Amino acid position 1298 (e.g., G1298R)⁵
Amino acid position 1302 (e.g., E1302X)⁵
Amino acid position 1311 (e.g., Y1311X)⁵⁷
Amino acid position 1316 (e.g., T1316Lfs*64)¹⁵
Amino acid position 1321 (e.g., S1321N)⁵⁷
Intron 4 ((+3)A > C)¹
IVS4 − 74A > T⁸⁹
Splice site mutation 3′ Intron 5 c.3901G > A⁵
Splice site mutation 5; Intron 7 c.6111G > A⁵
Splice site mutation IVS7 + 1G > A¹⁴
IVS7 + 5G > A⁴⁰
IVS8 + 1G > C⁷⁶
Splice site mutation 5′ Intron 9 c.9081delG⁵
Splice site mutation 5′ Intron 9 c.9081G > T⁵
Splice site mutation 5′ Intron 9 c.9081G > A⁵
Splice site mutation IVS9 + 1G > T¹⁴
Splice site mutation 3′ Intron 13 c.143513_1435-8del⁵
Splice site mutation IVS13del-13\hat{ }-8¹⁴
Splice site mutation 3′ Intron 16 c.20128T > G⁵
Splice site mutation IVS16 − 8T > G¹⁴
Splice site mutation 5′ Intron 18 c.21781G > T⁵
Splice site mutation 5′ Intron 18 c.21781G > A⁵
Splice site mutation 5′ Intron 18 c.21781G > C⁵
Splice site mutation 3′ Intron 18 c.21792A > G⁵
Splice site mutation IVS18 + 1G > A¹⁴
Splice site mutation 5′ Intron 19 c.2343 + 1G > T⁵
Splice site mutation 5′ Intron 19 c.2343 + 2T > C⁵
Splice site mutation IVS19 + 2T > C¹⁴
Splice site mutation IVS19 + 1G > A²²
Splice site mutation 3′ Intron 21 c.26112A > T⁵
IVS22 + 3A > G⁸⁹
IVS 23 − 8 G − A³⁶
IVS24 + 5G > A⁵¹
Splice site mutation 5′ Intron 24 c.32131delG⁵
IVS35 − 6C > G⁸⁹
Putative splice mutation 1198 − 1G > C¹⁷
Putative splice mutation 1810 − 3C > G¹⁷
Putative splice mutation 2178 + 1G > A¹⁷
Putative splice mutation 2344 − 1G > T¹⁷
Putative splice mutation c.2611 − 2A > T³⁹
Putative splice mutation 3213 + 1_3213 + 2delinsA¹⁷
c.-24C > A^{44,78}
c.76 13 G > T⁹
c.77 − 19T > A⁵²
c.90_93delGAAA¹⁸
c.124G > A⁶⁹
c.150 + 3 A > C¹⁰
174C > T⁵⁴
c.245T > C⁸⁷
c.249_250insT¹⁸
270T > C⁵⁴
402C > T⁵⁴
585G > C⁵⁴
c.611 + 1G > A⁷⁰
c.611 + 4A > G³⁶
c.612 − 15_-6del10bp⁵⁵
c.625A > C³¹
c.627 + 5G > T³¹
c.625A > C/c.627 + 5G > T³¹
696G > T⁵⁴
c. 784 + 1G > C⁴⁹
807T > C⁵⁴
c.886C > T³¹
c.890A > G⁵⁹
c.908 + 1G > A⁵⁷
c.908 + 5G > A⁵⁵
c.908delG⁵⁹
c.909 − 15A > G⁶⁶
957A > G⁵⁴
c.1084 − 2A > G⁵⁷
1145 1 bp deletion⁹⁰
1281C > T^{54,57}
c.1309 − 165C > T¹⁹
c.1434 + 174G > A¹⁹
c.1434 + 70C > T¹⁹
c.1530C > A⁵⁷
c.1587 − 1589delCTT³¹
c.1621A > C^{33,59}
c.1638 + 32T > C⁶⁶
c.1638 + 80C > T⁶⁶
1671C > T⁵⁴
1791G > T⁵⁴
1939delA¹⁴
c.2075 + 3A > G⁵³
c.2081T > A³¹
c.2093G > A⁶⁵
2098delA¹⁶
c.2138 − 8T > G⁶⁷
2142A > G⁵⁴
c.2178 + 1G > T^{36,39}
c.2179 − 17C > A⁶⁶
c.2344 − 157T > G⁶⁶
c.2344 − 17T > C⁶⁶
c.2417G > A⁷⁸
c.2541delG⁸⁷
c.2620C > T^{32,33}
c.2815 − 8A > G⁵⁵
c.3003A > G³⁷
c.3084A > G^{48,54}
c.3213 + 4 A > G^{9,37}
c.3213 + 5 G > A⁹
c.3268C > T⁷⁵
3285A > G⁵⁴
c.3382C > T⁷⁵
3435A > G⁵⁴
c.3491delT⁷²
c.3589C > T⁵⁷
c.3765(+1 + 5)del5⁴²
c.3766 − 34A > G⁶⁶
c.3767 − 3768insC⁶
c.3770delA⁶⁷
c.3826C > T⁷²
c.3846C > T⁵⁷
c.3929delG⁶⁷
c.*236A > G⁶⁶
1145delC⁸
Ex13_Ex17del⁸²

TABLE 4
Selected ABCB11 Mutations Associated with PFIC-2

Amino acid position 1 (e.g., M1V)⁹
Amino acid position 4 (e.g., S4X)⁶⁴
Amino acid position 19 (e.g., G19R)⁵⁶
Amino acid position 25 (e.g., S25X)¹⁴
Amino acid position 26 (e.g., Y26Ifs*7)³⁸
Amino acid position 50 (e.g., L50S)^{7,57}
Amino acid position 52 (e.g., R52W)²⁶
Amino acid position 58 (e.g., D58N)⁶²
Amino acid position 62 (e.g., M62K)⁹
Amino acid position 66 (e.g., S66N)¹⁷
Amino acid position 68 (e.g., C68Y)⁴¹
Amino acid position 93 (e.g., Y93S)¹³
Amino acid position 101 (e.g., Q101Dfs*8)⁹
Amino acid position 107 (e.g., C107R)³⁶
Amino acid position 112 (e.g., I112T)⁹
Amino acid position 114 (e.g., W114R)^{2,9}
Amino acid position 129 (e.g., C129Y)²⁵
Amino acid position 135 (e.g., E135K¹³, E135L¹⁷)
Amino acid position 167 (e.g., A167V⁷, A167T^{9,17})
Amino acid position 182 (e.g., I182K)⁹
Amino acid position 183 (e.g., M183V⁸, M183T⁹)
Amino acid position 225 (e.g., T225P)⁵⁷
Amino acid position 226 (e.g., S226L)⁹
Amino acid position 232 (e.g., L232Cfs*9)⁹
Amino acid position 233 (e.g., L233S)⁸⁶
Amino acid position 238 (e.g., G238V)^{2,7}
Amino acid position 242 (e.g., T242I)⁷
Amino acid position 245 (e.g., I245Tfs*26)⁵⁷
Amino acid position 256 (e.g., A256G)⁹
Amino acid position 260 (e.g., G260D)⁵⁷
Amino acid position 284 (e.g., V284L)⁷
Amino acid position 297 (e.g., E297G)^{2,7}
Amino acid position 303 (e.g., R303K⁸, R303M⁶³, R303fsX321⁸³)
Amino acid position 304 (e.g., Y304X)²⁶
Amino acid position 312 (e.g., Q312H)⁷
Amino acid position 313 (e.g., R313S)⁷
Amino acid position 314 (e.g., W314X)⁵⁷
Amino acid position 318 (e.g., K318Rfs*26)²⁹
Amino acid position 327 (e.g., G327E)⁷
Amino acid position 330 (e.g., V330X)²⁴
Amino acid position 336 (e.g., C336S)^{2,7}
Amino acid position 337 (e.g., Y337H)²¹
Amino acid position 342 (e.g., W342G)⁵⁰
Amino acid position 354 (e.g., R354X)⁹
Amino acid position 361 (e.g., Q361X)⁵⁷
Amino acid position 366 (e.g., V366D)⁵⁷
Amino acid position 386 (e.g., G386X)³⁴
Δ Amino acid positions 383-389⁵⁷
Amino acid position 387 (e.g., R387H)⁹
Amino acid position 390 (e.g., A390P)⁷
Amino acid position 410 (e.g., G410D)⁷
Amino acid position 413 (e.g., L413W)⁷
Amino acid position 415 (e.g., R415X)⁴²
Amino acid position 420 (e.g., I420T)⁹
Amino acid position 454 (e.g., V454X)⁴⁹
Amino acid position 455 (e.g., G455E)⁹
Amino acid position 461 (e.g., K461E)^{2,7}
Amino acid position 463 (e.g., T463I)⁷
Amino acid position 466 (e.g., Q466K)⁷
Amino acid position 470 (e.g., R470Q⁷, R470X⁹)
Amino acid position 472 (e.g., Y472X¹⁴, Y472C²⁷)
Amino acid position 475 (e.g., C475X)²⁹
Amino acid position 481 (e.g., V481E)⁷
Amino acid position 482 (e.g., D482G)^{2,7}
Amino acid position 484 (e.g., H484Rfs*5)⁹
Amino acid position 487 (e.g., R487H², R487P⁸⁴)
Amino acid position 490 (e.g., N490D)⁷
Amino acid position 493 (e.g., W493X)⁸
Amino acid position 498 (e.g., I498T)⁷
Amino acid position 501 (e.g., V501G)⁶⁸
Amino acid position 512 (e.g., I512T)⁷
Amino acid position 515 (e.g., N515T⁷, N515D⁶⁴)
Amino acid position 516 (e.g., I516M)¹⁷
Amino acid position 517 (e.g., R517H)⁷
Amino acid position 520 (e.g., R520X)⁵⁷
Amino acid position 523 (e.g., A523G)¹³
Amino acid position 528 (e.g., I528X)⁹
Amino acid position 540 (e.g., F540L)⁴⁶
Amino acid position 541 (e.g., I541L⁷, I541T¹⁷)
Amino acid position 548 (e.g., F548Y)⁷
Amino acid position 549 (e.g., D549V)⁹
Amino acid position 554 (e.g., E554K)²¹
Amino acid position 559 (e.g., M559T)⁵⁷
Amino acid position 562 (e.g., G562D)⁷
Amino acid position 570 (e.g., A570T⁷, A570V²⁶)
Amino acid position 575 (e.g., R575X², R575Q²¹)
Amino acid position 588 (e.g., A588V)⁷
Amino acid position 591 (e.g., N591S)^{9,17}
Amino acid position 593 (e.g., S593R)^{2,7}
Amino acid position 597 (e.g., V597V⁹, V597L¹³)
Amino acid positions 591 and 597 (e.g., N591S + V597V)⁹
Amino acid position 603 (e.g., K603K)⁵⁵
Amino acid position 609 (e.g., H609Hfs*46)²⁶
Amino acid position 610 (e.g., I610Gfs*45)⁹
Amino acid position 615 (e.g., H615R)²⁶
Amino acid position 625 (e.g., T625Nfs*5)²⁶
Amino acid position 627 (e.g., I627T)⁷
Amino acid position 636 (e.g., E636G)²
Amino acid position 669 (e.g., I669V)²⁶
Amino acid position 698 (e.g., R609H)⁹
Amino acid positions 112 and 698 (e.g., I112T + R698H)⁹
Amino acid position 699 (e.g., S699P)⁹
Amino acid position 766 (e.g., G766R)²⁴
Amino acid position 806 (e.g., G806G)⁵⁵
Amino acid position 824 (e.g., G824E)⁴²
Amino acid position 832 (e.g., R832C^{7,26}, R832H⁴¹)
Amino acid position 842 (e.g., D842G)²
Amino acid position 859 (e.g., T859R)⁷
Amino acid position 865 (e.g., A865V)⁴⁵
Amino acid position 877 (e.g., G877R)⁵⁶
Amino acid position 893 (e.g., A893V)⁵⁷
Amino acid position 901 (e.g., S901R)¹⁷
Amino acid position 903 (e.g., V903G)⁵⁷
Δ Amino acid position 919¹²
Amino acid position 928 (e.g., R928X)^{15,21}
Amino acid position 930 (e.g., K930Efs*79¹⁰, K930Efs*49²⁶)
Amino acid position 948 (e.g., R948C)^{7,26}
Amino acid position 979 (e.g., N979D)⁷
Amino acid position 982 (e.g., G982R)^{2,7}
Amino acid positions 444 and 982 (e.g., V444A + G982R)³⁸
Amino acid position 1001 (e.g., R1001R)⁹
Amino acid position 1003 (e.g., G1003R)²⁴
Amino acid position 1004 (e.g., G1004D)^{2,7}
Amino acid position 1027 (e.g., S1027R)²⁶
Amino acid position 1028 (e.g., A1028A)¹⁰
Amino acid position 1032 (e.g., G1032R)¹²
Amino acid position 1041 (e.g., Y1041X)⁹
Amino acid position 1050 (e.g., R1050C)⁵⁷
Amino acid position 1053 (e.g., Q1053X)⁵⁷
Amino acid position 1055 (e.g., L1055P)³⁶
Amino acid position 1057 (e.g., R1057X)²
Amino acid position 1058 (e.g., Q1058Hfs*38⁹, Q1058fs*38¹⁷)
Amino acid position 1061 (e.g., I1061Vfs*34)⁹
Amino acid position 1083 (e.g., C1083Y)⁴⁷
Amino acid position 1090 (e.g., R1090X)²
Amino acid position 1099 (e.g., L1099Lfs*38)²⁶
Amino acid position 1100 (e.g., S1100Qfs*38)¹³
Amino acid position 1110 (e.g., A1110E)⁷
Amino acid position 1116 (e.g., G1116R⁷, G1116F^{9,17}, G1116E³⁶)
Amino acid position 1128 (e.g., R1128C)^{7,13}
Amino acid position 1131 (e.g., D1131V)²⁷
Amino acid position 1144 (e.g., S1144R)⁷
Amino acid position 1153 (e.g., R1153C^{2,7}, R1153H^{7,26})
Amino acid position 1154 (e.g., S1154P)⁷
Amino acid position 1173 (e.g., N1173D)⁵⁷
Amino acid position 1192 (e.g., A1192Efs*50)⁹
Amino acid position 1198 (e.g., H1198R)²⁷
Amino acid position 1210 (e.g., T1210P⁷, T1210F⁵⁷)
Amino acid position 1211 (e.g., N1211D)⁷
Amino acid position 1212 (e.g., V1212F)³⁶
Amino acid position 1231 (e.g., R1231W⁷, R1223Q⁷)
Amino acid position 1232 (e.g., A1232D)¹⁷
Amino acid position 1235 (e.g., R1235X)¹²
Amino acid position 1242 (e.g., L1242I)⁷
Amino acid position 1256 (e.g., T1256fs*1296)⁸³
Amino acid position 1268 (e.g., R1268Q)^{2,7}
Amino acid position 1302 (e.g. E1302X)⁵⁷
Amino acid position 1311 (e.g., Y1311X)⁵⁷
Amino acid position 1316 (e.g., T1316Lfs*64)¹⁵
Intron 4 ((+3)A > C)¹
Splice site mutation IVS7 + 1G > A¹⁴
IVS8 + 1G > C⁷⁶
Splice site mutation IVS9 + 1G > T¹⁴
Splice site mutation IVS13del-13\hat{ }-8¹⁴
Splice site mutation IVS16 − 8T > G¹⁴
Splice site mutation IVS18 + 1G > A¹⁴
Splice site mutation IVS19 + 2T > C¹⁴
IVS 23 − 8 G − A³⁶
IVS24 + 5G > A⁵¹
Putative splice mutation 1198 − 1G > C¹⁷
Putative splice mutation 1810 − 3C > G¹⁷
Putative splice mutation 2178 + 1G > A¹⁷
Putative splice mutation 2344 − 1G > T¹⁷
Putative splice mutation 3213 + 1_3213 + 2delinsA¹⁷
c.-24C > A⁷⁸
c.76 13 G > T⁹
c.77 − 19T > A⁵²
c.90_93delGAAA¹⁸
c.124G > A⁶⁹
c.150 + 3 A > C¹⁰
c.249_250insT¹⁸
c.611 + 1G > A⁸⁴
c.611 + 4A > G³⁶
c.612 − 15_-6del10bp⁵⁵
c.625A > C³¹
c.627 + 5G > T³¹
c.625A > C/c.627 + 5G > T³¹
c.886C > T³¹
c.890A > G⁵⁹
c.908 + 1G > A⁵⁷
c.908 + 5G > A⁵⁵
c.908delG⁵⁹
1273 1 bp deletion⁹¹
c.1084 − 2A > G⁵⁷
c.1445A > G⁵⁹
c.1587-1589delCTT³¹
c.1621A > C⁵⁹
1939delA¹⁴
c.2081T > A³¹
2098delA¹⁶
c.2343 + 1 G > T⁸⁰
c.2178 + 1G > T³⁶
c.2417G > A⁷⁸
c.2620C > T³²
c.2815 − 8A > G⁵⁵
c.3003A > G³⁷
c.3213 + 4 A > G^{9,37}
c.3213 + 5 G > A⁹
c.3268C > T⁷⁵
c.3382C > T⁷⁵
c.3765(+1 + 5)del5⁴²
c.3767-3768insC⁶
1145delC⁸
Ex13_Ex17del⁸²
^{A} A mutation to ‘X’ denotes an early stop codon

### References for Tables 3 and 4

¹ Noe et al., J Hepatol. 2005, vol. 43(3), p. 536-543.
² Lam et al., Am J Physiol Cell Physiol. 2007, vol. 293(5), p. C1709-16.
³ Stindt et al., Liver Int. 2013, vol. 33(10), p. 1527-1735.
⁴ Gao et al., Shandong Yiyao 2012, vol. 52(10), p. 14-16.
⁵ Strautnieks et al., Gastroenterology. 2008, vol. 134(4), p. 1203-1214.
⁶ Kagawa et al., Am J Physiol Gastrointest Liver Physiol. 2008, vol. 294(1), p. G58-67.
⁷ Byrne et al., Hepatology. 2009, vol. 49(2), p. 553-567.
⁸ Chen et al., J Pediatr. 2008, vol. 153(6), p. 825-832.
⁹ Davit-Spraul et al., Hepatology 2010, vol. 51(5), p. 1645-1655.
¹⁰ Droge et al., Sci Rep. 2016, vol. 6: 24827.
¹¹ Lang et al., Pharmacogenet Genomics. 2007, vol. 17(1), p. 47-60.
¹² Ellinger et al., World J Gastroenterol. 2017, vol. 23(29), p.:5295-5303.
¹³ Vitale et al., J Gastroenterol. 2018, vol. 53(8), p. 945-958.
¹⁴ Knisely et al., Hepatology. 2006, vol. 44(2), p. 478-86.
¹⁵ Ellis et al., Hepatology. 2018, vol. 67(4), p. 1531-1545.
¹⁶ Lam et al., J Hepatol. 2006, vol. 44(1), p. 240-242.
¹⁷ Varma et al., Hepatology 2015, vol. 62(1), p. 198-206.
¹⁸ Treepongkaruna et al., World J Gastroenterol. 2009, vol. 15(34), p. 4339-4342.
¹⁹ Zarenezhad et al., Hepatitis Monthly: 2017, vol. 17(2); e43500.
²⁰ Hayashi et al., Hepatol Res. 2016, vol. 46(2), p. 192-200.
²¹ Guorui et al., Linchuang Erke Zazhi 2013, vol. 31(10), 905-909.
²² van Mil et al., Gastroenterology. 2004, vol. 127(2), p. 379-384.
²³ Anzivino et al., Dig Liver Dis. 2013, vol. 45(3), p. 226-232.
²⁴ Park et al., World J Gastroenterol. 2016, vol. 22(20), p. 4901-4907.
²⁵ Imagawa et al., J Hum Genet. 2018, vol. 63(5), p. 569-577.
²⁶ Giovannoni et al., PLoS One. 2015, vol. 10(12): e0145021.
²⁷ Hu et al., Mol Med Rep. 2014, vol. 10(3), p. 1264-1274.
²⁸ Lang et al,. Drug Metab Dispos. 2006, vol. 34(9), p. 1582-1599.
²⁹ Masahata et al., Transplant Proc. 2016, vol. 48(9), p. 3156-3162.
³⁰ Holz et al., Hepatol Commun. 2018, vol. 2(2), p. 152-154.
³¹ Li et al., Hepatology International 2017, vol. 11, No. 1, Supp. Supplement 1, pp. S180. Abstract Number: OP284.
³² Francalanci et al., Laboratory Investigation 2011, vol. 91, Supp. SUPPL. 1, pp. 360A. Abstract Number: 1526.
³³ Francalanci et al., Digestive and Liver Disease 2010, vol. 42, Supp. SUPPL. 1, pp. S16. Abstract Number: T.N.5.
³⁴ Shah et al., J Pediatr Genet. 2017, vol. 6(2), p. 126-127.
³⁵ Gao et al., Hepatitis Monthly 2017, vol. 17(10), e55087/1-e55087/6.
³⁶ Evason et al., Am J Surg Pathol. 2011, vol. 35(5), p. 687-696.
³⁷ Davit-Spraul et al., Mol Genet Metab. 2014, vol. 113(3), p. 225-229.
³⁸ Maggiore et al., J Hepatol. 2010, vol. 53(5), p. 981-6.
³⁹ McKay et al., Version 2. F1000Res. 2013; 2: 32. DOI: 10.12688/f1000research.2-32.v2
⁴⁰ Liu et al., Pediatr Int. 2013, vol. 55(2), p. 138-144.
⁴¹ Waisbourd-Zinman et al., Ann Hepatol. 2017, vol. 16(3), p. 465-468.
⁴² Griffin, et al., Canadian Journal of Gastroenterology and Hepatology 2016, vol. 2016. Abstract Number: A200. Meeting Info: 2016 Canadian Digestive Diseases Week, CDDW 2016. Montreal, QC, United States. 26-2-2016-29-2-2016
⁴³ Qiu et al., Hepatology 2017, vol. 65(5), p. 1655-1669.
⁴⁴ Imagawa et al., Sci Rep. 2017, 7:41806.
⁴⁵ Kang et al., J Pathol Transl Med. 2019-5-16. doi: 10.4132/jptm.2019.05.03. [Epub ahead of print]
⁴⁶ Takahashi et al., Eur J Gastroenterol Hepatol. 2007, vol. 19(11), p. 942-6.
⁴⁷ Shimizu et al., Am J Transplant. 2011, vol. 11(2), p. 394-398.
⁴⁸ Krawczyk et al., Ann Hepatol. 2012, vol. 11(5), p. 710-744.
⁴⁹ Sharma et al., BMC Gastroenterol. 2018, vol. 18(1), p. 107.
⁵⁰ Sattler et al., Journal of Hepatology 2017, vol. 66, No. 1, Suppl. S, pp. S177. Meeting Info.: International Liver Congress/52nd Annual Meeting of the European-Association-for-the-Study-of-the-Liver. Amsterdam, NETHERLANDS. Apr. 19-23, 2017. European Assoc Study Liver.
⁵¹ Jung et al., J Pediatr Gastroenterol Nutr. 2007, vol. 44(4), p. 453-458.
⁵² Sciveres. Digestive and Liver Disease 2010, vol. 42, Supp. SUPPL. 5, pp. S329. Abstract Number: C018. Meeting Info: 17th National Congress SIGENP. Pescara, Italy. 7-10-2010-9-10-2010
⁵³ Sohn et al., Pediatr Gastroenterol Hepatol Nutr. 2019, vol. 22(2), p. 201-206.
⁵⁴ Ho et al., Pharmacogenet Genomics. 2010, vol. 20(1), p. 45-57.
⁵⁵ Wang et al., Hepatol Res. 2018, vol. 48(7), p. 574-584.
⁵⁶ Shaprio et al., J Hum Genet. 2010, vol. 55(5), p. 308-313.
⁵⁷ Bounford. University of Birmingham. Dissertation Abstracts International, (2016) Vol. 75, No. 1C. Order No.: AAI10588329. ProQuest Dissertations & Theses.
⁵⁸ Stolz et al., Aliment Pharmacol Ther. 2019, vol. 49(9), p. 1195-1204.
⁵⁹ Jankowska et al., J Pediatr Gastroenterol Nutr. 2014, vol. 58(1), p. 92-95.
⁶⁰ Kim. Journal of Pediatric Gastroenterology and Nutrition 2016, vol. 62, Supp. SUPPL. 1, pp. 620. Abstract Number: H-P-045. Meeting Info: 49th Annual Meeting of the European Society for Paediatric Gastroenterology, Hepatology and Nutrition, ESPGHAN 2016. Athens, Greece. 25 May 2016-28-5-2016.
⁶¹ Pauli-Magnus et al., Hepatology 2003, vol. 38, No. 4 Suppl. 1, pp. 518A. print. Meeting Info.: 54th Annual Meeting of the American Association for the Study of Liver Diseases. Boston, MA, USA. Oct. 24-28, 2003. American Association for the Study of Liver Diseases.
⁶² Li et al., Hepatology International 2017, vol. 11, No. 1, Supp. Supplement 1, pp. S362. Abstract Number: PP0347. Meeting Info: 26th Annual Conference of the Asian Pacific Association for the Study of the Liver, APASL 2017. Shanghai, China. 15-2-2017-19-2-2017.
⁶³ Rumbo et al., Transplantation 2018, vol. 102, No. 7, Supp. Supplement 1, pp. S848. Abstract Number: P.752. Meeting Info: 27th International Congress of The Transplantation Society, T T S 2018. Madrid, Spain. 30-6-2018-5-7-2018.
64 Lee et al., Pediatr Gastroenterol Hepatol Nutr. 2017, vol. 20(2), p. 114-123.
⁶⁵ Sherrif et al., Liver international: official journal of the International Association for the Study of the Liver 2013, vol. 33, No. 8, pp. 1266-1270.
⁶⁶ Blackmore et al., J Clin Exp Hepatol. 2013, vol. 3(2), p. 159-161.
⁶⁷ Matte et al., J Pediatr Gastroenterol Nutr. 2010, vol. 51(4), p. 488-493.
⁶⁸ Lin et al., Zhongguo Dang Dai Er Ke Za Zhi. 2018, vol. 20(9), p. 758-764.
⁶⁹ Harmanci et al., Experimental and Clinical Transplantation 2015, vol. 13, Supp. SUPPL. 2, pp. 76. Abstract Number: P62. Meeting Info: 1st Congress of the Turkic World Transplantation Society. Astana, Kazakhstan. 20-5-2015-22-5-2015.
⁷⁰ Herbst et al., Mol Cell Probes. 2015, vol. 29(5), p. 291-298.
⁷¹ Moghadamrad et al., Hepatology. 2013, vol. 57(6), p. 2539-2541.
⁷² Holz et al., Zeitschrift fur Gastroenterologie 2016, vol. 54, No. 8. Abstract Number: KV275. Meeting Info: Viszeralmedizin 2016, 71. Jahrestagung der Deutschen Gesellschaft fur Gastroenterologie, Verdauungs-und Stoffwechselkrankheiten mit Sektion Endoskopie—10. Herbsttagung der Deutschen Gesellschaft fur Allgemein-und Viszeralchirurgie. Hamburg, Germany. 21-9-2016-24 Sep. 2016.
⁷³ Wang et al., PLoS One. 2016; vol. 11(4): e0153114.
⁷⁴ Hao et al., International Journal of Clinical and Experimental Pathology 2017, vol. 10(3), p. 3480-3487.
⁷⁵ Arnell et al., J Pediatr Gastroenterol Nutr. 2010, vol. 51(4), p. 494-499.
⁷⁶ Sharma et al., Indian Journal of Gastroenterology 2017, vol. 36, No. 1, Supp. Supplement 1, pp. A99. Abstract Number: M-20. Meeting Info: 58th Annual Conference of the Indian Society of Gastroenterology, ISGCON 2017. Bhubaneswar, India. 14-12-2017-17-12-2017.
⁷⁷ Beausejour et al., Can J Gastroenterol. 2011, vol. 25(6), p. 311-314.
⁷⁸ Imagawa et al., Journal of Pediatric Gastroenterology and Nutrition 2016, vol. 63, Supp. Supplement 2, pp. S51. Abstract Number: 166. Meeting Info: World Congress of Pediatric Gastroenterology, Hepatology and Nutrition 2016. Montreal, QC, Canada. 5-10-2016-8 Oct. 2016.
⁷⁹ Peng et al., Zhonghua er ke za zhi (Chinese journal of pediatrics) 2018, vol. 56, No. 6, pp. 440-444.
⁸⁰ Tibesar et al., Case Rep Pediatr. 2014, vol. 2014: 185923.
⁸¹ Ng et al., Journal of Pediatric Gastroenterology and Nutrition 2018, vol. 66, Supp. Supplement 2, pp. 860. Abstract Number: H-P-127. Meeting Info: 51st Annual Meeting European Society for Paediatric Gastroenterology, Hepatology and Nutrition, ESPGHAN 2018. Geneva, Switzerland. 9 May 2018-12-5-2018.
⁸² Wong et al., Clin Chem. 2008, vol. 54(7), p. 1141-1148.
⁸³ Pauli-Magnus et al., J Hepatol. 2005, vol. 43(2), p. 342-357.
⁸⁴ Jericho et al., Journal of Pediatric Gastroenterology and Nutrition. 60, vol. 3, p. 368-374.
⁸⁵ Scheimann et al., Gastroenterology 2007, vol. 132, No. 4, Suppl. 2, pp. A452. Meeting Info.: Digestive Disease Week Meeting/108th Annual Meeting of the American-Gastroenterological-Association. Washington, DC, USA. May 19-24, 2007. Amer Gastroenterol Assoc; Amer Assoc Study Liver Dis; Amer Soc Gastrointestinal Endoscopy; Soc Surg Alimentary Tract.
⁸⁶ Jaquotot-Haerranz et al., Rev Esp Enferm Dig. 2013, vol. 105(1), p. 52-54.
⁸⁷ Khosla et al., American Journal of Gastroenterology 2015, vol. 110, No. Suppl. 1, pp. S397. Meeting Info.: 80th Annual Scientific Meeting of the American-College-of-Gastroenterology. Honolulu, HI, USA. Oct. 16-21, 2015.
⁸⁸ Droge et al., J Hepatol. 2017, vol. 67(6), p. 1253-1264.
⁸⁹ Liu et al., Liver International 2010, vol. 30(6), p. 809-815.
⁹⁰ Chen et al., Journal of Pediatrics 2002, vol. 140(1), p. 119-124.
⁹¹ U.S. Pat. No. 9,295,677

In some embodiments, the mutation in ABCB11 is selected from A167T, G238V, V284L, E297G, R470Q, R470X, D482G, R487H, A570T, N591S, A865V, G982R, R1153C, and R1268Q.

Provided are methods of treating PFIC (e.g., PFIC-1 and PFIC-2) in a subject that includes performing an assay on a sample obtained from the subject to determine whether the subject has a mutation associated with PFIC (e.g., a ATP8B1, ABCB11, ABCB4, TJP2, NR1H4 or Myo5b mutation), and administering (e.g., specifically or selectively administering) a therapeutically effective amount of a compound of formula (1), or a pharmaceutically acceptable salt thereof, to the subject determined to have a mutation associated with PFIC. In some embodiments, the mutation is an ATP8B1 or ABCB11 mutation. For example, a mutation as provided in any one of Tables 1-4. In some embodiments, the mutation in ATP8B1 is selected from L127P, G308V, T456M, D554N, F529del, 1661T, E665X, R930X, R952X, R1014X, and G1040R. In some embodiments, the mutation in ABCB11 is selected from A167T, G238V, V284L, E297G, R470Q, R470X, D482G, R487H, A570T, N591S, A865V, G982R, R1153C, and R1268Q.

Also provided are methods for treating PFIC (e.g., PFIC-1 and PFIC-2) in a subject in need thereof, the method comprising: (a) detecting a mutation associated with PFIC (e.g., a ATP8B1, ABCB11, ABCB4, TJP2, NR1H4 or Myo5b mutation) in the subject; and (b) administering to the subject a therapeutically effective amount of crystal modification 1 of odevixibat. In some embodiments, methods for treating PFIC can include administering a therapeutically effective amount of a compound of formula (I), or a pharmaceutically acceptable salt thereof, to a subject having a mutation associated with PFIC (e.g., an ATP8B1, ABCB11, ABCB4, TJP2, NR1H4 or Myo5b mutation).

In some embodiments, the mutation is an ATP8B1 or ABCB11 mutation. For example, a mutation as provided in any one of Tables 1-4. In some embodiments, the mutation in ATP8B1 is selected from L127P, G308V, T456M, D554N, F529del, 1661T, E665X, R930X, R952X, R1014X, and G1040R. In some embodiments, the mutation in ABCB11 is selected from A167T, G238V, V284L, E297G, R470Q, R470X, D482G, R487H, A570T, N591S, A865V, G982R, R1153C, and R1268Q.

In some embodiments, the subject is determined to have a mutation associated with PFIC in a subject or a biopsy sample from the subject through the use of any art recognized tests, including next generation sequencing (NGS). In some embodiments, the subject is determined to have a mutation associated with PFIC using a regulatory agency-approved, e.g., FDA-approved test or assay for identifying a mutation associated with PFIC in a subject or a biopsy sample from the subject or by performing any of the non-limiting examples of assays described herein. Additional methods of diagnosing PFIC are described in Gunaydin, M. et al., Hepat Med. 2018, vol. 10, p. 95-104, incorporated by reference in its entirety herein.

In some embodiments, the treatment of PFIC (e.g., PFIC-1 or PFIC-2) decreases the level of serum bile acids in the subject. In some embodiments, the level of serum bile acids is determined by, for example, an ELISA enzymatic assay or the assays for the measurement of total bile acids as described in Danese et al., PLoS One. 2017, vol. 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of crystal modification 1 of odevixibat. In some embodiments, the treatment of PFIC includes treatment of pruritus.

Thus, in one embodiment, the invention relates to crystal modification 1 of odevixibat described herein for use in the treatment or prevention of a disease or disorder as listed above.

In another embodiment, the invention relates to the use of crystal modification 1 of odevixibat described herein in the manufacture of a medicament for the treatment or prevention of a disease or disorder as listed above.

In yet another embodiment, the invention relates to a method of treatment or prevention of a disease or disorder as listed above in a warm-blooded animal, comprising administering a therapeutically effective amount of crystal modification 1 of odevixibat described herein to a warm-blooded animal in need of such treatment and/or prophylaxis.

Another aspect of the invention relates to a pharmaceutical composition comprising a therapeutically effective amount of crystal modification 1 of odevixibat described herein, in association with a pharmaceutically acceptable diluent or carrier.

The pharmaceutical composition may further comprise at least one other active substance, such as an active substance selected from an IBAT inhibitor; an enteroendocrine peptide or enhancer thereof; a dipeptidyl peptidase-IV inhibitor; a biguanidine; an incretin mimetic; a thiazolidinone; a PPAR agonist; a HMG Co-A reductase inhibitor; a bile acid binder; a TGR5 receptor modulator; a member of the prostone class of compounds; a guanylate cyclase C agonist; a 5-HT4 serotonin agonist; or a pharmaceutically acceptable salt of any one these active substances. Examples of such combinations are also described in WO2012/064268.

Crystal modification 1 of odevixibat can be administered to a warm-blooded animal at a unit dose within the range of about 0.01 to 1.0 mg/kg, such as about 0.01 to 0.5 mg/kg, or such as about 0.01 to 0.2 mg/kg, and this can provide a therapeutically effective dose. A unit dose form, such as a tablet or capsule, can contain about 0.1 to 20 mg of active ingredient, such as about 0.1 to 10 mg, or such as about 0.2 to 5 mg, or such as about 0.2 to 1.0 mg. The daily dose can be administered as a single dose or divided into one, two, three or more unit doses. An orally administered daily dose of odevixibat is preferably within about 0.1 to 50 mg, more preferably within about 0.1 to 20 mg, such as within about 0.2 to 10 mg, or such as within about 0.2 to 5.0 mg.

Pharmaceutical formulations of odevixibat may comprise a therapeutically effective amount of crystal modification 1 of odevixibat, and one or more pharmaceutically acceptable excipients. The excipients may e.g. include fillers, binders, disintegrants, glidants and lubricants. In general, pharmaceutical compositions may be prepared in a conventional manner using conventional excipients.

In some embodiments, the pharmaceutical formulation is a multiparticulate formulation containing low doses of crystal modification 1 of odevixibat. Such a formulation enables weight-based dosing and may be particularly suitable for administering to paediatric patients. In some embodiments, the pharmaceutical formulation is a paediatric formulation.

In some embodiment, the particles are small enough that they can be sprinkled onto food and easily swallowed. In some embodiments, the particles can be swallowed without causing a perception of grittiness. In some embodiments, the particles do not give the patient an urge to chew the particles.

In some embodiments, each particle comprises a core and a coating layer surrounding the core. The core of each particle may be a pellet, a granule, a minitablet, a bead, a microparticle or a microsphere. The active pharmaceutical ingredient may be in the core or in the coating layer. In some embodiments, the coating layer of each particle comprises the active pharmaceutical ingredient, while the core of each particle does not comprise the active pharmaceutical ingredient.

The cores may be orally dispersible and comprise soluble ingredients such as a sugar (e.g., sucrose) or a soluble polymer (e.g. hydroxypropyl methylcellulose) or may be non-orally dispersible and comprise non-soluble ingredients such as a non-soluble polymer (e.g., microcrystalline cellulose). In some embodiments, the cores are microcrystalline cellulose spheres.

The coating layer can further comprise a film-forming polymer, such as a cellulose-based polymer, a polysaccharide-based polymer, an N-vinylpyrrolidone-based polymer, an acrylate, an acrylamide, or copolymers thereof. Examples of suitable film-forming polymers include polyvinyl alcohol (PVA), polyvinyl acetate phthalate (PVAP), polyethylene glycol (PEG), polyvinylpyrrolidone (PVP), methacrylic acid copolymers, starch, hydroxypropyl starch, chitosan, shellac, methyl cellulose, hydroxypropyl cellulose (HPC), low-substituted hydroxypropyl cellulose, hydroxypropyl methylcellulose (HPMC; or hypromellose), hydroxypropyl methylcellulose acetate succinate (HPMCAS), hydroxypropyl methylcellulose phthalate (HPMCP), cellulose acetate phthalate (CAP), cellulose acetate trimellitate (CAT), as well as combinations thereof, such as a mixture of methyl cellulose and hydroxypropyl methylcellulose (metolose). In some embodiments, the coating layer comprises a film-forming polymer selected from the group consisting of hydroxypropyl methylcellulose, polyvinyl alcohol (PVA), polyethylene glycol (PEG), starch, hydroxypropyl starch and hydroxypropyl cellulose (HPC).

The coating layer may optionally comprise one or more additional ingredients, such as a plasticizer (e.g. polyethylene glycol, triacetin or triethyl citrate), an anti-tack agent (e.g. talc or magnesium stearate) or a colouring agent (e.g. titanium dioxide, iron oxides, riboflavin or turmeric).

The dosage required for the therapeutic or prophylactic treatment will depend on the route of administration, the severity of the disease, the age and weight of the patient and other factors normally considered by the attending physician when determining the individual regimen and dosage levels appropriate for a particular patient.

### Definitions

The term “crystal modification” refers to a crystalline solid phase of an organic compound. A crystal modification can be either a solvate or an ansolvate.

The term “solvate” refers to a crystalline solid phase of an organic compound, which has solvent (i.e., solvent molecules) incorporated into its crystal structure. A “hydrate” is a solvate wherein the solvent is water.

The term “sesquihydrate” refers to a hydrate containing about 1.5 moles of water associated with the crystal per mole of organic compound (i.e., a 1.5 hydrate). As used herein, a sesquihydrate includes from about 1.2 to about 1.8, more preferably from about 1.3 to about 1.7, more preferably from about 1.4 to about 1.6 and even more preferably from about 1.45 to about 1.55 moles of water associated with each mole of odevixibat in a crystal. The amount of water calculated herein excludes water adsorbed to the surface of the crystal.

The term “mixed solvate” refers to a crystalline solid phase of an organic compound, which has two or more different solvent molecules incorporated into its crystal structure. One of the at least two solvent molecules may be water.

The term “isostructural solvate” refers to a crystalline solid phase of an organic compound, wherein the crystalline solid phase can accommodate different solvents without distortion of the crystalline structure.

The term “slurry” refers to a saturated solution to which an excess of solid is added, thereby forming a mixture of solid and saturated solution.

As used herein, the term “void volumes” refers to channels, layers or other more or less isolated voids in the crystal structure.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The crystallinity of a crystalline sample of odevixibat may be measured e.g. by X-Ray Powder Diffraction (XRPD) methods or by Differential Scanning Calorimetry (DSC) methods, such as the method disclosed in the experimental section. When reference is made herein to a crystalline compound, preferably the crystallinity as measured by DSC methods is greater than about 70%, such as greater than about 80%, particularly greater than about 90%, more particularly greater than about 95%. In some embodiments, the degree of crystallinity as measured by DSC methods is greater than about 98%. In some embodiments, the degree of crystallinity as measured by DSC methods is greater than about 99%. The % crystallinity refers to the percentage by weight of the total sample mass which is crystalline.

Preferably a crystal modification according to the invention is substantially free from other crystal modifications of the compound. Preferably, the described crystal modifications of odevixibat include less than, for example, about 20%, about 15%, about 10%, about 5%, about 3%, or particularly, less than about 1% by weight of other crystal modifications of odevixibat. Thus, preferably, the solid phase purity of the described crystal modifications of odevixibat is greater than about 80%, greater than about 85%, greater than about 90%, greater than about 95%, greater than about 97%, or particularly greater than about 99%.

The invention will now be described by the following examples which do not limit the invention in any respect. All cited documents and references mentioned herein are incorporated by reference in their entireties.

### Abbreviations

DMF dimethylformamide
DMSO dimethyl sulfoxide
EtOH ethanol
MecOH methanol
RH relative humidity
2-PrOH 2-propanol

### EXPERIMENTAL METHODS

### X-Ray Powder Diffraction (XRPD) Analysis

Analyses were performed at 22° C. on a PANalytical X'Pert Pro diffractometer equipped with a Cu long fine focus X-ray tube and a PIXcel detector. Automatic divergence and anti-scatter slits were used together with 0.02 rad Soller slits and a Ni-filter. Dry samples were smeared onto cut Silicon Zero Background Holders (ZBH) and analysed between 2-40° in 2-theta with an analysis time of 17 minutes. All slurry samples were dripped on tempered porous Alumina filter substrates and analysed twice as they dried, first with a one minute 16-second scan (2-30° in 2-theta) and then a 7-minute scan (2-30° in 2-theta). A final 17-minute scan was performed when the sample had dried for several hours.

The samples were spun during analysis in order to increase the randomness of the samples. The following experimental settings were used:
Tube tension and current: 40 kV, 50 mA
Wavelength alpha1 (CuKα1): 1.5406 A
Wavelength alpha2 (CuKα2): 1.5444 A
Wavelength alpha1 and alpha2 mean (CuKα): 1.5418 A

It is known in the art that an X-ray powder diffraction pattern may be obtained having one or more measurement errors depending on measurement conditions (such as equipment, sample preparation or machine used). In particular, it is generally known that intensities in an XRPD pattern may fluctuate depending on measurement conditions and sample preparation. For example, persons skilled in the art of XRPD will realise that the relative intensities of peaks may vary according to the orientation of the sample under the test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern presented herein is not to be construed as absolute and any crystalline form that provides a powder diffraction pattern substantially identical to those disclosed herein fall within the scope of the present disclosure (for further information, see R. Jenkins and R. L. Snyder, “Introduction to X-ray powder diffractometry”, John Wiley & Sons, 1996).

### Thermogravimetric Analysis (TGA)

The analyses were performed on a Mettler TGA/SDTA 851e, equipped with a Julabo FP40 cooler. 1-10 mg of sample was weighed into 100 μL Al-cups and flushed with dry nitrogen gas during the analysis. Two different methods were used: in the “standard scan” the sample was scanned from 25 to 200° C. with a scan rate of 10° C./minute, and in the “careful scan” the sample was kept at 25° C. for 30 minutes and was then scanned from 25 to 100° C. with a scan rate of 10° C./minute.

### Dynamic Vapor Sorption (DVS)

DVS measurements were performed with an SPS11-100n “Sorptions Prufsystem” from ProUmid (formerly “Projekt Messtechnik”), August-Nagel-Str. 23, 89079 Ulm (Germany). About 20 mg of sample was used. Humidity change rates of 5% per hour were used. The sample was placed on an aluminum or platinum holder on top of a microbalance and allowed to equilibrate at 0% RH before starting the pre-defined humidity program:
(1) 5 h at 0% RH
(2) 0→95% RH (5%/h); 5 h at 95% RH
(3) 95→0% RH (5%/h); 5 h at 0% RH
(4) 0→95% RH (5%/h); 5 h at 95% RH
(5) 95→0% RH (5%/h); 5 h at 0% RH

### High-Performance Liquid Chromatography (HPLC)

Analyses were performed on an Agilent, Series 1100, equipped with an Agilent 1260 Infinity degasser. Column: Waters XSelcet CHS C18 (150×3 mm, 3.5 am); Mobile phase A: 0.1% formic acid in water, mobile phase B: 0.1% formic acid in acetonitrile; Gradient 45% to 90% B; flow rate 0.425 mL/min; Acquisition time 35 minutes; Run time 42 minutes; Wave length: 283 nm; Column temperature 20° C. The Chromeleon Version 6.8 software was used.

### Differential Scanning Calorimetry (DSC)

Experiments were performed using a TA Instruments Q2000 Differential Scanning Calorimeter. The DCS crucible used was a TZero aluminum pan with pinhole (diameter ≥0.2 mm) in the lid. A dry nitrogen purge at a constant flow rate of 50 mL/min was maintained in the DSC cell throughout the measurement.

### EXAMPLES

### Example 1

### Preparation of Crystal Modification 1

Absolute alcohol (100.42 kg) and crude odevixibat (18.16 kg) were charged to a 250-L GLR with stirring under nitrogen atmosphere. Purified water (12.71 kg) was added and the reaction mass was stirred under nitrogen atmosphere at 25±5° C. for 15 minutes. Stirring was continued at 25±5° C. for 3 to 60 minutes, until a clear solution had formed. The solution was filtered through a 5.0 p SS cartridge filter, followed by a 0.2 I PP cartridge filter and then transferred to a clean reactor. Purified water (63.56 kg) was added slowly over a period of 2 to 3 hours at 25±5° C., and the solution was seeded with crystal modification 1 of odevixibat. The solution was stirred at 25±5° C. for 12 hours. During this time, the solution turned turbid. The precipitated solids were filtered through centrifuge and the material was spin dried for 30 minutes. The material was thereafter vacuum dried in a Nutsche filter for 12 hours. The material was then dried in a vacuum tray drier at 25±5° C. under vacuum (550 mm Hg) for 10 hours and then at 30±5° C. under vacuum (550 mm Hg) for 16 hours. The material was isolated as an off-white crystalline solid. The isolated crystalline material was milled and stored in LDPE bags.

An overhydrated sample was analyzed with XRPD and the diffractogram is shown in FIG. 2. Another sample was dried at 50° C. in vacuum and thereafter analysed with XRPD. The diffractogram of the dried sample is shown in FIG. 1.

The diffractograms for the drying of the sample are shown in FIGS. 3 and 4 for 2θ ranges 5-13° and 18-25°, respectively (overhydrated sample at the bottom and dry sample at the top).

### Example 2

Preparation of Crystal Modification 2A from Ethanol and Water

105.9 mg of odevixibat were weighed into a 1 mL Chromacol vessel. A magnetic stir bar and 1.0 mL of an ethanol:water 70:30% v/v mixture were added and the vessel was closed with a crimped cap. The resulting slurry was then left stirred at 25° C. for 1 week.

The wet sample was analyzed with XRPD and the diffractogram is shown in FIG. 6. Upon drying of the sample, it transformed into crystal modification 1.

### Example 3

Preparation of Crystal Modification 2A from Acetone and Water

27.0 mg of odevixibat were weighed into a 1 mL Chromacol vessel. A magnetic stir bar and 0.5 mL of a acetone:water 50:50% v/v mixture were added and the vessel was closed with a crimped cap. The resulting slurry was then left stirred at 3° C. for 2 weeks.

The wet sample was analyzed with XRPD and the diffractogram is shown in FIG. 7. Upon drying of the sample, it transformed into crystal modification 1.

### Example 4

Preparation of Crystal Modification 2A from 2-Propanol and Water

27.4 mg of odevixibat were weighed into a 1 mL Chromacol vessel. A magnetic stir bar and 0.5 mL of a 2-propanol:water 50:50% v/v mixture were added and the vessel was closed with a crimped cap. The resulting slurry was then left stirred at 3° C. for 2 weeks.

The wet sample was analyzed with XRPD and the diffractogram is shown in FIG. 8. Upon drying of the sample, it transformed into crystal modification 1.

### Example 5

Preparation of Crystal Modification 2A from 1,4-Dioxane and Water

31.6 mg of odevixibat were weighed into a 1 mL Chromacol vessel. A magnetic stir bar and 0.5 mL of a 1,4-dioxane:water 50:50% v/v mixture were added and the vessel was closed with a crimped cap. The resulting slurry was then left stirred at 3° C. for 2 weeks.

The wet sample was analyzed with XRPD and the diffractogram is shown in FIG. 9. Upon drying of the sample, it transformed into crystal modification 1.

### Example 6

Preparation of Crystal Modification 2B from Methanol

103.9 mg of odevixibat were weighed into a 1 mL Chromacol vessel. A magnetic stir bar and 0.9 mL of methanol was added and the vessel was closed with a crimped cap. The resulting slurry was then left stirred at 22° C. for 1 week.

The wet sample was analyzed with XRPD and the diffractogram is shown in FIG. 9. Upon drying of the sample, it transformed into crystal modification 1.

### Example 7

Preparation of Crystal Modification 2B from Acetonitrile and Water

20.2 mg of odevixibat were dissolved in 1.5 mL acetonitrile. To the stirred solution, 2.5 mL water was added as an antisolvent. Within 20-30 minutes a slurry had precipitated.

The wet sample was analyzed with XRPD and the diffractogram is shown in FIG. 10. Upon drying of the sample, it transformed into crystal modification 1.

### Example 8

Preparation of Crystal Modification 2C from DMSO and Water

29.8 mg of odevixibat were weighed into a 1 mL Chromacol vessel. A magnetic stir bar and 0.5 mL of a DMSO:water 50:50% v/v mixture were added and the vessel was closed with a crimped cap. The resulting slurry was then left stirred at 3° C. for 2 weeks.

The wet sample was analyzed with XRPD and the diffractogram is shown in FIG. 12. Upon drying of the sample, it transformed into crystal modification 1.

### Example 9

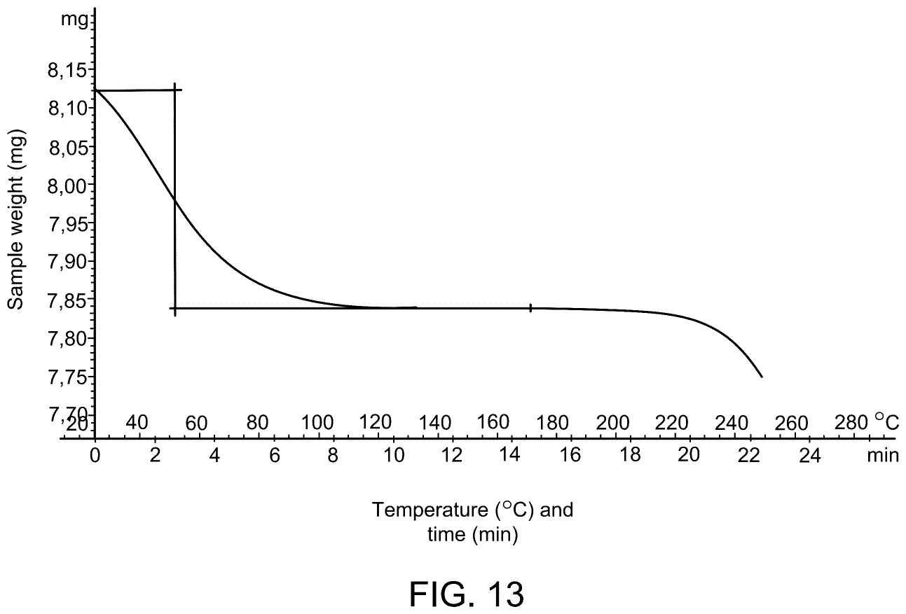

Analysis of the water and solvent content of crystal modifications 1 and 2 Karl-Fischer analysis of crystals of modification 1 showed a water content of 3.4% w/w. Thermal gravimetric analysis (TGA) of the same material showed a total mass loss of 3.5% (see FIG. 13). These similar findings indicate that crystal modification 1 contains 1.5 moles of water per mole of odevixibat, corresponding to a 1.5 hydrate.

The water and solvent content in crystal modification 2 were analysed by using samples prepared from a slurry of odevixibat in ethanol:water (60:40% v/v) that had been allowed to equilibrate during 3 days. Form 2 had formed according to XRPD. Slurry samples were taken from the slurry to Porous Plates and then stored in a desiccator with ethanol:water (60:40% v/v) and equilibrated at least overnight. Plates were taken out and dried in air for a certain time (5-30 minutes), and then analysed with a fast scan on XRPD (1 min 16 s) to verify the crystal form. Some samples contained crystal modification 2 and were still very wet, whereas crystal modification 1 already started to appear in the drier samples. Karl-Fischer analysis of the dried samples of crystal modification 2 indicated a water content of slightly more than 4% w/w. Thermal gravimetric analysis of the very wet samples of crystal modification 2 showed that these samples initially lost a lot of mass. A change in drying rate was thereafter observed, which probably indicates the start of the transformation from modification 2 to modification 1. After performing several experiments, a mass loss of approximately 12% w/w could be determined for the transformation of modification 2 to modification 1. Since dry modification 1 is a sesquihydrate (see FIG. 13), the total mass loss of approximately 12% (w/w) for the transformation of crystal modification 2 to crystal modification 1 would correspond to a loss of two moles of ethanol and 0.5 moles of water.

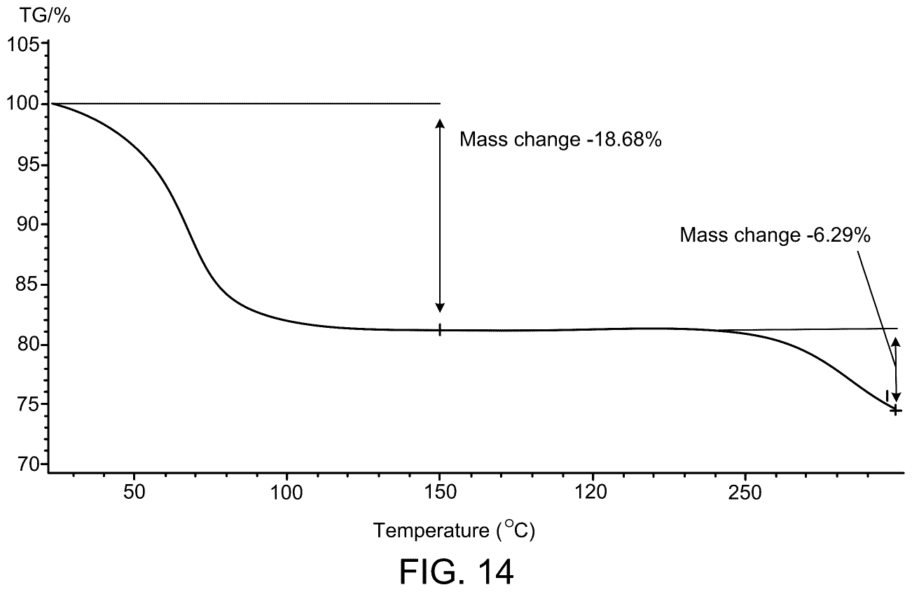

In another experiment, a sample of crystal modification 1 was kept in a dessicator and exposed to the vapour phase of a 60:40 (% v/v) mixture of ethanol and water for 4 days at room temperature. Thermal gravimetric analysis of the sample showed a mass loss of about 18.7% (see FIG. 14). The mass loss begins readily at the beginning of the experiment. Further examination of the sample by 1H-NMR suggested that the ethanol content corresponded to about 2.7 equivalents and the water content to about 1.9 equivalents.

### Example 10

### Dynamic Vapor Sorption Analysis of Crystal Modification 1

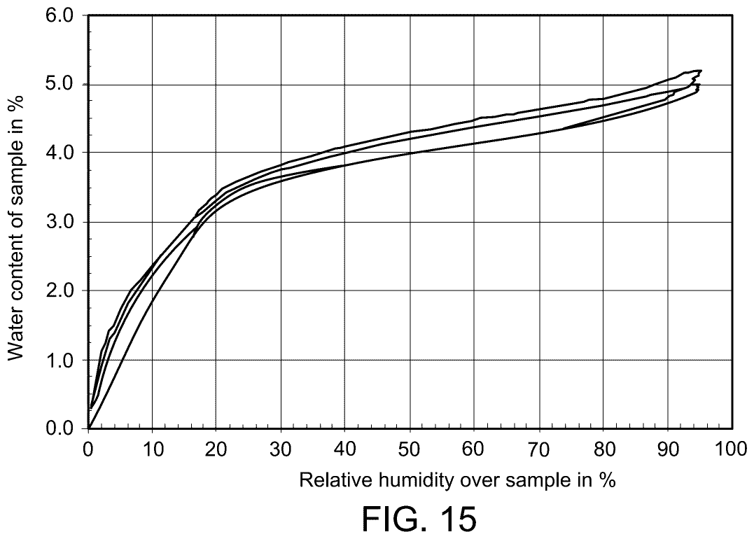

The water uptake of crystal modification 1 was measured using dynamic vapour sorption (DVS). The measurements demonstrate that the water content is reversibly dependent on the environmental humidity with maximum uptakes of about 5.0% (w/w) at 95% RH, as shown in FIG. 15.

After drying the sample at 0% RH and increasing the relative humidity, most of the water was taken back up to about 25% RH. This corresponds to a water content of about 3.5% (w/w). An additional 1.5% (w/w) of water was then taken up when the humidity was increased up to 95% RH. The sorption/desorption process shows minimal hysteresis. XRPD analysis has shown that the hydrate structure is almost completely restored at 20% RH and is completely restored at 30% RH. Crystal modification 1 therefore seems to require about 3.5% (w/w) of water, which corresponds to a sesquihydrate. The further water uptake at higher relative humidities does not change the structure any further. Crystal modification 1 is therefore likely a slightly hygroscopic sesquihydrate that can take up additional 1.5% (w/w) of water at elevated relative humidity in the range of 30-95% RH.

### Example 11

### Stability Testing

Samples of amorphous odevixibat (purity ˜91%) and of crystal modification 1 of odevixibat (purity >99%; crystallinity 100%) were stored in a closed container under air at 80° C. The amount of odevixibat in the samples was determined by HPLC at the beginning of the experiment, and was again determined after 1, 2 and 4 weeks. The results are shown in the table below. After 4 weeks of storage, the amorphous sample showed 0.3% decomposition, whereas the purity of the crystalline sample had not changed.

Odevixibat content (%)

| Time (weeks) | Amorphous odevixibat | crystal modification 1 |
|---|---|---|
| 0 | 91.1 | 99.13 |
| 1 | 90.9 | 99.15 |
| 2 | 91.04 | 99.18 |
| 4 | 90.8 | 99.24 |

### Example 12

### Determination of Crystalline Fraction by Differential Scanning Calorimetry

This method quantifies the crystalline fraction of odevixibat drug substance in partially crystalline samples. The quantification is based on the assumption that partially crystalline samples are binary mixtures of the crystalline hydrate and the amorphous phase of odevixibat. The crystalline fraction is quantified based on the melting enthalpy of an anhydrous form. This anhydrous form is the dehydrated hydrate which spontaneously and reproducibly forms by drying the hydrate at elevated temperature.

5-6 mg of a sample of a crystalline or partially crystalline sample of odevixibat was accurately weighed into a DSC crucible which was then closed with a perforated lid using a suitable press. The total weight of the DSC crucible (pan+lid+sample) was noted and the total weight of the crucible was again determined after the DSC test. The weight loss during the DSC test must not be more than 5%.

The DSC test consists of three cycles:
Cycle 1: an increase in temperature from 20° C. to 120° C. at a scanning rate of 5° C./min;
Cycle 2: a decrease in temperature from 120° C. to 80° C. at a scanning rate of 10° C./min; and
Cycle 3: an increase in temperature from 80° C. to 200° C. at a scanning rate of 10° C./min.

The first scan cycle dries the sample and thereby converts the hydrate form into a dehydrated hydrate (an anhydrous form). In the second scan cycle, the sample is cooled down to obtain a stable baseline in the subsequent heat-up for signal integration. The melting enthalpy is determined in the third scan cycle, where the sample is heated through the melting of the anhydrous form.

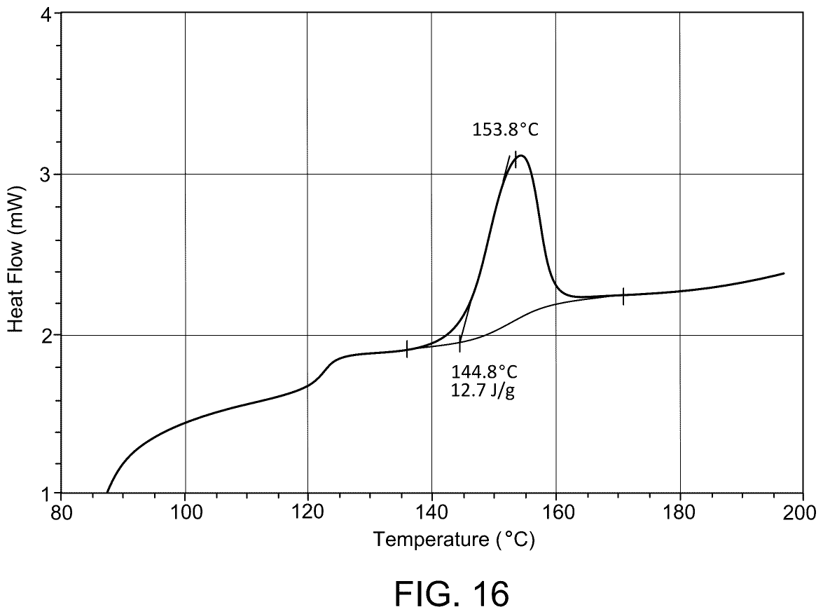

The endothermic event due to melting appears in the temperature range of 140-165° C. The peak must be integrated over a sigmoidal tangent baseline using the Sig Tangent integration function of the TA Universal Analysis software. The integration should start at a temperature between 130° C. and 140° C., and end at a temperature between 165° C. and 175° C., depending on the actual baseline. The glass transition of the amorphous part may appear in the temperature range of 120-130° C., depending on the actual amorphous fraction (see FIG. 16 for an example). If an irregular baseline does not allow the integration, it should be assessed whether the drying of the sample was incomplete.

The evaluation of the melting enthalpy is done by using the dry weight of the sample, which is obtained by subtracting the total weight of the DSC crucible (pan+lid+sample) after the DSC test from the total weight of the crucible before the test. The percent weight loss during the DSC scan, which is the difference between the initial weight and the dry weight divided by the initial weight, must not be more than 5%; otherwise the crystalline content of the sample cannot be calculated.

The crystalline fraction expressed in weight percent is to be calculated from the melting enthalpy (ΔHₛₐₘₚₗₑ) based on the following formula. The value shall be given on an integer number.

$$\% crystalline content = \frac{Δ {H}_{sample} + 1.1626}{0.2815}$$

### Example 13

### Effect of Drying on the Crystallinity of Crystal Modification 1

In these experiments, crystal modification 2 was obtained after slurring of crystal modification 1 in a 6:4 mixture of ethanol/water; the obtained wet material was thereafter stored in a desiccator under ethanol/water (6:4) vapor for two months.

Samples of crystal modification 2 were then dried using different drying techniques, in order to see the impact of drying on the crystallinity of crystal modification 1. The dried samples were analyzed using XRPD (samples were prepared in an ambient air atmosphere) and the results are shown in the table below. The results suggest that crystal modification 1 is obtained by rehydration of the dehydrated form, which is obtained by drying of crystal modification 2 under vacuum or under nitrogen flow. When crystal modification 2 is stored at ambient conditions, the ethanol-water exchange seems to be very low.

| Drying conditions | Results |
|---|---|
| Vacuum (<5 mbar), room temperature. | Crystal modification 1 |
| Nitrogen flow, room temperature | Crystal modification 1 |
| Ambient conditions | Poorly crystalline crystal |
| | modification 1 |

### Example 14

### Effect of Solvent on Crystallinity of Crystal Modification 2

Crystal modification 1 was suspended in a 30:70 (% v/v) mixture of ethanol and water (sample A) or in a 70:30 (% v/v) mixture of ethanol and water (sample B) at room temperature. After stirring overnight, filtration was conducted and the recovered wet samples were submitted for XRPD (transmission). The XRPD patterns for both samples essentially corresponded to crystal modification 2, but some slight peak shifts were observed between the two samples, possibly due to the difference in ethanol content of the two samples.

Both samples were then subjected to air drying at room temperature and retested by XRPD. In both cases, crystal modification 1 was obtained, but based on the peak resolution in the XRPD patterns the sample obtained from the 70:30 (% v/v) mixture of ethanol and water appeared considerably more crystalline.

DSC measurements were conducted on the air-dried samples. It was found that sample A, obtained from the mixture containing 30% ethanol, was less crystalline than sample B, obtained from the mixture containing 70% ethanol. An enthalpy of fusion of 25.7 J/g was found for sample A which corresponds to 95% of crystallinity. For sample B, an enthalpy of 28.9 J/g was found, which corresponds to more than 100% crystallinity.

1.-27. (canceled)

28. A method for treating a cholestatic liver disease comprising orally administering to a subject in need of such treatment a therapeutically effective amount of a pharmaceutical formulation comprising a crystalline hydrate of odevixibat, or a pharmaceutically acceptable salt thereof.

29. The method of claim 28, wherein the crystalline hydrate of odevixibat comprises from about 0 to about 2 moles of water associated with the crystal per mole of odevixibat.

30. The method of claim 28, wherein the crystalline hydrate of odevixibat is a sesquihydrate.

31. The method of claim 28, wherein the crystalline hydrate of odevixibat has an XRPD pattern, obtained with CuKα1-radiation, with peaks at ° 2θ positions 5.6±0.2, 6.7±0.2 and/or 12.1±0.2.

32. The method of claim 31, wherein the crystalline hydrate of odevixibat has an XRPD pattern, obtained with CuKα1-radiation, with specific peaks at ° 2θ positions 5.6±0.2, 6.7±0.2 and 12.1±0.2 and one or more of the characteristic peaks: 4.1±0.2, 4.6±0.2, 9.3±0.2, 9.4±0.2 and 10.7±0.2.

33. The method of claim 28, wherein the cholestatic liver disease is cholestatic pruritus.

34. The method of claim 28, wherein the cholestatic liver disease is selected from the group consisting of: biliary atresia, Alagille syndrome (ALGS), progressive familial intrahepatic cholestasis (PFIC), primary biliary cirrhosis (PBC), and primary sclerosing cholangitis (PSC).

35. The method of claim 34, wherein the cholestatic liver disease is biliary atresia.

36. The method of claim 35, wherein the biliary atresia is post-Kasai biliary atresia or post-liver transplantation biliary atresia.

37. The method of claim 34, wherein the cholestatic liver disease is PFIC.

38. The method of claim 37, wherein the PFIC is PFIC Type 1, PFIC Type 2, PFIC Type 3, non-specified PFIC, post-biliary diversion PFIC, or post-liver transplant PFIC.

39. The method of claim 34, wherein the cholestatic liver disease is ALGS.

40. The method of claim 28, wherein the subject is a pediatric subject.

41. The method of claim 28, wherein the subject is administered a daily dose of about 0.01 mg/kg to about 0.2 mg/kg of the crystalline hydrate of odevixibat, or a pharmaceutically acceptable salt thereof.

42. The method of claim 28, wherein the subject is administered a daily dose of about 0.2 mg to about 10 mg of the crystalline hydrate of odevixibat, or a pharmaceutically acceptable salt thereof.

43. The method of claim 28, wherein the subject is administered a daily dose of about 0.2 mg to about 5 mg of the crystalline hydrate of odevixibat, or a pharmaceutically acceptable salt thereof.

44. The method of claim 28, wherein the pharmaceutical formulation comprises a plurality of particles, each particle comprising a core and a coating layer surrounding the core, wherein the coating layer comprises the crystalline hydrate of odevixibat, or a pharmaceutically acceptable salt thereof.

45. The method of claim 44, wherein a unit dose of the pharmaceutical formulation comprises about 0.2 mg to about 5 mg of the crystalline hydrate of odevixibat, or a pharmaceutically acceptable salt thereof.

46. The method of claim 44, wherein a unit dose of the pharmaceutical formulation comprises about 0.2 mg to about 1.0 mg of the crystalline hydrate of odevixibat, or a pharmaceutically acceptable salt thereof.

47. The method of claim 44, wherein the core comprises microcrystalline cellulose.

48. The method of claim 44, wherein the coating layer further comprises a film-forming polymer.

49. The method of claim 48, wherein the film-forming polymer is hydroxypropyl methylcellulose.

50. The method of claim 44, wherein the plurality of particles is contained within a capsule.


SEQUENCE LISTING (4 sequences)

SEQ ID NO: 1 - protein, 1251 aa, Homo sapiens
MSTERDSETTFDEDSQPNDEVVPYSDDETEDELDDQGSAVEPEQNRVNREAEENREPFRKECTWQVKANDRKYHEQPHFMNTKFLCIKESKYANNAIKTYKYNAFTFIPMNLFEQFKRAANLYFLALLILQAVPQISTLAWYTTLVPLLVVLGVTAIKDLVDDVARHKMDKEINNRTCEVIKDGRFKVAKWKEIQVGDVIRLKKNDFVPADILLLSSSEPNSLCYVETAELDGETNLKFKMSLEITDQYLQREDTLATFDGFIECEEPNNRLDKFTGTLFWRNTSFPLDADKILLRGCVIRNTDFCHGLVIFAGADTKIMKNSGKTRFKRTKIDYLMNYMVYTIFVVLILLSAGLAIGHAYWEAQVGNSSWYLYDGEDDTPSYRGFLIFWGYIIVLNTMVPISLYVSVEVIRLGQSHFINWDLQMYYAEKDTPAKARTTTLNEQLGQIHYIFSDKTGTLTQNIMTFKKCCINGQIYGDHRDASQHNHNKIEQVDFSWNTYADGKLAFYDHYLIEQIQSGKEPEVRQFFFLLAVCHTVMVDRTDGQLNYQAASPDEGALVNAARNFGFAFLARTQNTITISELGTERTYNVLAILDFNSDRKRMSIIVRTPEGNIKLYCKGADTVIYERLHRMNPTKQETQDALDIFANETLRTLCLCYKEIEEKEFTEWNKKFMAASVASTNRDEALDKVYEEIEKDLILLGATAIEDKLQDGVPETISKLAKADIKIWVLTGDKKETAENIGFACELLTEDTTICYGEDINSLLHARMENQRNRGGVYAKFAPPVQESFFPPGGNRALIITGSWLNEILLEKKTKRNKILKLKFPRTEEERRMRTQSKRRLEAKKEQRQKNFVDLACECSAVICCRVTPKQKAMVVDLVKRYKKAITLAIGDGANDVNMIKTAHIGVGISGQEGMQAVMSSDYSFAQFRYLQRLLLVHGRWSYIRMCKFLRYFFYKNFAFTLVHFWYSFFNGYSAQTAYEDWFITLYNVLYTSLPVLLMGLLDQDVSDKLSLRFPGLYIVGQRDLLFNYKRFFVSLLHGVLTSMILFFIPLGAYLQTVGQDGEAPSDYQSFAVTIASALVITVNFQIGLDTSYWTFVNAFSIFGSIALYFGIMFDFHSAGIHVLFPSAFQFTGTASNALRQPYIWLTIILAVAVCLLPVVAIRFLSMTIWPSESDKIQKHRKRLKAEEQWQRRQQVFRRGVSTRRSAYAFSHQRGYADLISSGRSIRKKRSPLDAIVADGTAEYRRTGDS

SEQ ID NO: 2 - DNA, 3756 bp, Homo sapiens
  source 1..3756: mol_type = genomic DNA
atgagtacagaaagagactcagaaacgacatttgacgaggattctcagcctaatgacgaagtggttccctacagtgatgatgaaacagaagatgaacttgatgaccaggggtctgctgttgaaccagaacaaaaccgagtcaacagggaagcagaggagaaccgggagccattcagaaaagaatgtacatggcaagtcaaagcaaacgatcgcaagtaccacgaacaacctcactttatgaacacaaaattcttgtgtattaaggagagtaaatatgcgaataatgcaattaaaacatacaagtacaacgcatttacctttataccaatgaatctgtttgagcagtttaagagagcagccaatttatatttcctggctcttcttatcttacaggcagttcctcaaatctctaccctggcttggtacaccacactagtgcccctgcttgtggtgctgggcgtcactgcaatcaaagacctggtggacgatgtggctcgccataaaatggataaggaaatcaacaataggacgtgtgaagtcattaaggatggcaggttcaaagttgctaagtggaaagaaattcaagttggagacgtcattcgtctgaaaaaaaatgattttgttccagctgacattctcctgctgtctagctctgagcctaacagcctctgctatgtggaaacagcagaactggatggagaaaccaatttaaaatttaagatgtcacttgaaatcacagaccagtacctccaaagagaagatacattggctacatttgatggttttattgaatgtgaagaacccaataacagactagataagtttacaggaacactattttggagaaacacaagttttcctttggatgctgataaaattttgttacgtggctgtgtaattaggaacaccgatttctgccacggcttagtcatttttgcaggtgctgacactaaaataatgaagaatagtgggaaaaccagatttaaaagaactaaaattgattacttgatgaactacatggtttacacgatctttgttgttcttattctgctttctgctggtcttgccatcggccatgcttattgggaagcacaggtgggcaattcctcttggtacctctatgatggagaagacgatacaccctcctaccgtggattcctcattttctggggctatatcattgttctcaacaccatggtacccatctctctctatgtcagcgtggaagtgattcgtcttggacagagtcacttcatcaactgggacctgcaaatgtactatgctgagaaggacacacccgcaaaagctagaaccaccacactcaatgaacagctcgggcagatccattatatcttctctgataagacggggacactcacacaaaatatcatgacctttaaaaagtgctgtatcaacgggcagatatatggggaccatcgggatgcctctcaacacaaccacaacaaaatagagcaagttgattttagctggaatacatatgctgatgggaagcttgcattttatgaccactatcttattgagcaaatccagtcagggaaagagccagaagtacgacagttcttcttcttgctcgcagtttgccacacagtcatggtggataggactgatggtcagctcaactaccaggcagcctctcccgatgaaggtgccctggtaaacgctgccaggaactttggctttgccttcctcgccaggacccagaacaccatcaccatcagtgaactgggcactgaaaggacttacaatgttcttgccattttggacttcaacagtgaccggaagcgaatgtctatcattgtaagaaccccagaaggcaatatcaagctttactgtaaaggtgctgacactgttatttatgaacggttacatcgaatgaatcctactaagcaagaaacacaggatgccctggatatctttgcaaatgaaactcttagaaccctatgcctttgctacaaggaaattgaagaaaaagaatttacagaatggaataaaaagtttatggctgccagtgtggcctccaccaaccgggacgaagctctggataaagtatatgaggagattgaaaaagacttaattctcctgggagctacagctattgaagacaagctacaggatggagttccagaaaccatttcaaaacttgcaaaagctgacattaagatctgggtgcttactggagacaaaaaggaaactgctgaaaatataggatttgcttgtgaacttctgactgaagacaccaccatctgctatggggaggatattaattctcttcttcatgcaaggatggaaaaccagaggaatagaggtggcgtctacgcaaagtttgcacctcctgtgcaggaatctttttttccacccggtggaaaccgtgccttaatcatcactggttcttggttgaatgaaattcttctcgagaaaaagaccaagagaaataagattctgaagctgaagttcccaagaacagaagaagaaagacggatgcggacccaaagtaaaaggaggctagaagctaagaaagagcagcggcagaaaaactttgtggacctggcctgcgagtgcagcgcagtcatctgctgccgcgtcacccccaagcagaaggccatggtggtggacctggtgaagaggtacaagaaagccatcacgctggccatcggagatggggccaatgacgtgaacatgatcaaaactgcccacattggcgttggaataagtggacaagaaggaatgcaagctgtcatgtcgagtgactattcctttgctcagttccgatatctgcagaggctactgctggtgcatggccgatggtcttacataaggatgtgcaagttcctacgatacttcttttacaaaaactttgcctttactttggttcatttctggtactccttcttcaatggctactctgcgcagactgcatacgaggattggttcatcaccctctacaacgtgctgtacaccagcctgcccgtgctcctcatggggctgctcgaccaggatgtgagtgacaaactgagcctccgattccctgggttatacatagtgggacaaagagacttactattcaactataagagattctttgtaagcttgttgcatggggtcctaacatcgatgatcctcttcttcatacctcttggagcttatctgcaaaccgtagggcaggatggagaggcaccttccgactaccagtcttttgccgtcaccattgcctctgctcttgtaataacagtcaatttccagattggcttggatacttcttattggacttttgtgaatgctttttcaatttttggaagcattgcactttattttggcatcatgtttgactttcatagtgctggaatacatgttctctttccatctgcatttcaatttacaggcacagcttcaaacgctctgagacagccatacatttggttaactatcatcctggctgttgctgtgtgcttactacccgtcgttgccattcgattcctgtcaatgaccatctggccatcagaaagtgataagatccagaagcatcgcaagcggttgaaggcggaggagcagtggcagcgacggcagcaggtgttccgccggggcgtgtcaacgcggcgctcggcctacgccttctcgcaccagcggggctacgcggacctcatctcctccgggcgcagcatccgcaagaagcgctcgccgcttgatgccatcgtggcggatggcaccgcggagtacaggcgcaccggggacagctga

SEQ ID NO: 3 - protein, 1321 aa, Homo sapiens
MSDSVILRSIKKFGEENDGFESDKSYNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 4 - DNA, 3966 bp, Homo sapiens
  source 1..3966: mol_type = genomic DNA
atgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggtcattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga